(19) United States
(12) Patent Application Publication
(10) Pub. No.: US 2024/0404255 A1
(43) Pub. Date: Dec. 5, 2024

(54) GENERATION OF ARTIFICIAL CONTRAST-ENHANCED RADIOLOGICAL IMAGES

(71) Applicant: BAYER AKTIENGESELLSCHAFT, Leverkusen (DE)

(72) Inventors: Matthias LENGA, Leverkusen (DE); Ivo Matteo BALTRUSCHAT, Leverkusen (DE); Parvaneh JANBAKHSHI, Leverkusen (DE); Felix Karl KREIS, Leverkusen (DE)

(21) Appl. No.: 18/678,323

(22) Filed: May 30, 2024

(30) Foreign Application Priority Data
Jun. 5, 2023 (EP) 23177301.1
Jul. 13, 2023 (EP) 23185296.3

(51) Int. Cl. G06V 10/774; A61K 49/10; G06V 10/82

(52) U.S. Cl. CPC G06V 10/774; A61K 49/105; A61K 49/108; G06V 10/82; G06V 2201/03

(57) ABSTRACT

The present disclosure is concerned with the technical field of generation of artificial contrast-enhanced radiological images.

### CROSS-REFERENCE TO RELATED APPLICATIONS

This application claims priority to European Patent Application No. 23177301.1, filed Jun. 5, 2023, and European Patent Application No. 23185296.3, filed Jul. 13, 2023, the contents of which are incorporated herein by reference in their entirety.

### FIELD OF THE DISCLOSURE

The present disclosure is concerned with the technical field of generation of artificial contrast-enhanced radiological images.

### BACKGROUND

WO 2019/074938 A1 discloses a method for reducing the amount of contrast agent in the generation of radiological images with the aid of an artificial neural network.

In the disclosed method, a training data set is created in a first step. The training data set comprises for each person of a multiplicity of persons i) a native radiological image (zero-contrast image), ii) a radiological image after administration of a small amount of contrast agent (low-contrast image) and iii) a radiological image after administration of a standard amount of contrast agent (full-contrast image). The standard amount is the amount recommended by the manufacturer and/or distributor of the contrast agent and/or the amount approved by a regulatory authority and/or the amount specified in a package leaflet for the contrast agent.

In a second step, an artificial neural network is trained to predict for each person of the training data set, on the basis of the native image and the image after administration of an amount of contrast agent smaller than the standard amount, an artificial radiological image showing an acquisition region after administration of the standard amount of contrast agent. The measured radiological image after administration of a standard amount of contrast agent is used in each case as reference (ground truth) in the training.

In a third step, the trained artificial neural network can be used to predict for a new person, on the basis of a native image and of a radiological image after administration of an amount of contrast agent smaller than the standard amount, an artificial radiological image showing the acquired region as it would look if a standard amount of contrast agent had been administered.

The method disclosed in WO 2019/074938 A1 has disadvantages.

The artificial neural network described in WO 2019/074938 A1 is a black box, i.e. it is not possible to track exactly what the artificial neural network is learning when it is being trained. It is unclear to what extent the artificial neural network is able to make predictions on the basis of data that had not been used in training. It is possible, on the basis of further data, to carry out a validation of the artificial neural network. Such validation data must be obtained (generated) in addition to the training data, but the available validation data will be unable to cover all situations that can occur when the trained artificial neural network is used later for prediction. This means there will often be uncertainty as to whether the trained artificial neural network is able to make meaningful/correct predictions for all inputted data.

In many countries a permit is necessary in order to employ a trained artificial neural network, such as is described in WO 2019/074938 A1, for the diagnosis of pathologies in human patients. The described circumstance that a trained artificial neural network represents a black box for which there is uncertainty as to whether the trained artificial neural network will often generate meaningful/correct predictions makes the approval process more difficult.

### SUMMARY

The present disclosure is dedicated to this and other problems.

The present disclosure provides in a first aspect a computer-implemented method for generating a synthetic contrast-enhanced radiological image, comprising the steps of:
providing a trained machine-learning model,
   where the trained machine-learning model has been trained on the basis of training data,
   where the training data for each reference object of a multiplicity of reference objects comprise as target data: (i) a first reference representation of a reference region of the reference object and a second reference representation of the reference region of the reference object as input data and (ii) a third reference representation of the reference region of the reference object,
   where the first reference representation represents the reference region without contrast agent or after administration of a first amount of a contrast agent,
   where the second reference representation represents the reference region after administration of a second amount of the contrast agent, the second amount being larger than the first amount,
   where the third reference representation represents the reference region after administration of a third amount of the contrast agent, the third amount being different from the second amount, preferably larger than the second amount,
   where the trained machine-learning model comprises two submodels: a first submodel and a second submodel, where the first submodel is configured and has been trained to determine, based on the first reference representation and second reference representation of a reference object, at least one model parameter for the second submodel and where the second submodel is configured to generate, based on the first reference representation and/or second reference representation and on the at least one model parameter determined by the first submodel, a synthetic third representation,
providing a first representation, where the first representation represents an examination region of an examination object without contrast agent or after administration of a first amount of a contrast agent,
providing a second representation, where the second representation represents the examination region of the examination object after administration of a second amount of the contrast agent, the second amount being larger than the first amount,
feeding the first representation and the second representation to the trained machine-learning model,
receiving from the trained machine-learning model a synthetic third representation of the examination region of the examination object, where the synthetic third representation represents the reference region after administration of a third amount of the contrast agent, the third amount being different from the second amount, preferably larger than the second amount,
outputting and/or storing the synthetic third representation and/or transmitting the synthetic third representation to a separate computer system.

The present disclosure further provides a computer system comprising:
a processor; and
a storage medium that stores a computer program configured to perform an operation when executed by the processor, said operation comprising the steps of:
   providing a trained machine-learning model,
      where the trained machine-learning model has been trained on the basis of training data,
      where the training data for each reference object of a multiplicity of reference objects comprise as target data: (i) a first reference representation of a reference region of the reference object and a second reference representation of the reference region of the reference object as input data and (ii) a third reference representation of the reference region of the reference object,
      where the first reference representation represents the reference region without contrast agent or after administration of a first amount of a contrast agent,
      where the second reference representation represents the reference region after administration of a second amount of the contrast agent, the second amount being larger than the first amount,
      where the third reference representation represents the reference region after administration of a third amount of the contrast agent, the third amount being different from the second amount, preferably larger than the second amount,
      where the trained machine-learning model comprises two submodels: a first submodel and a second submodel, where the first submodel is configured and has been trained to determine, based on the first reference representation and second reference representation of a reference object, at least one model parameter for the second submodel and where the second submodel is configured to generate, based on the first reference representation and/or second reference representation and on the at least one model parameter determined by the first submodel, a synthetic third representation,
   providing a first representation, where the first representation represents an examination region of an examination object without contrast agent or after administration of a first amount of a contrast agent,
   providing a second representation, where the second representation represents the examination region of the examination object after administration of a second amount of the contrast agent, the second amount being larger than the first amount,
   feeding the first representation and the second representation to the trained machine-learning model,
   receiving from the trained machine-learning model a synthetic third representation of the examination region of the examination object, where the synthetic third representation represents the reference region after administration of a third amount of the contrast agent, the third amount being different from the second amount, preferably larger than the second amount,
   outputting and/or storing the synthetic third representation and/or transmitting the synthetic third representation to a separate computer system.

The present disclosure further provides a computer program that can be loaded into a working memory of a computer system, where it causes the computer system to execute the following steps:
providing a trained machine-learning model,
   where the trained machine-learning model has been trained on the basis of training data,
   where the training data for each reference object of a multiplicity of reference objects comprise as target data: (i) a first reference representation of a reference region of the reference object and a second reference representation of the reference region of the reference object as input data and (ii) a third reference representation of the reference region of the reference object,
   where the first reference representation represents the reference region without contrast agent or after administration of a first amount of a contrast agent,
   where the second reference representation represents the reference region after administration of a second amount of the contrast agent, the second amount being larger than the first amount,
   where the third reference representation represents the reference region after administration of a third amount of the contrast agent, the third amount being different from the second amount, preferably larger than the second amount,
   where the trained machine-learning model comprises two submodels: a first submodel and a second submodel, where the first submodel is configured and has been trained to determine, based on the first reference representation and second reference representation of a reference object, at least one model parameter for the second submodel and where the second submodel is configured to generate, based on the first reference representation and/or second reference representation and on the at least one model parameter determined by the first submodel, a synthetic third representation,
providing a first representation, where the first representation represents an examination region of an examination object without contrast agent or after administration of a first amount of a contrast agent,
providing a second representation, where the second representation represents the examination region of the examination object after administration of a second amount of the contrast agent, the second amount being larger than the first amount,
feeding the first representation and the second representation to the trained machine-learning model,
receiving from the trained machine-learning model a synthetic third representation of the examination region of the examination object, where the synthetic third representation represents the reference region after administration of a third amount of the contrast agent, the third amount being different from the second amount, preferably larger than the second amount,
outputting and/or storing the synthetic third representation and/or transmitting the synthetic third representation to a separate computer system.

The present disclosure further provides for the use of a contrast agent in a radiological examination method, where the radiological examination method comprises the steps of:
providing a trained machine-learning model,
   where the trained machine-learning model has been trained on the basis of training data,
   where the training data for each reference object of a multiplicity of reference objects comprise as target data: (i) a first reference representation of a reference region of the reference object and a second reference representation of the reference region of the reference object as input data and (ii) a third reference representation of the reference region of the reference object,
   where the first reference representation represents the reference region without contrast agent or after administration of a first amount of the contrast agent,
   where the second reference representation represents the reference region after administration of a second amount of the contrast agent, the second amount being larger than the first amount,
   where the third reference representation represents the reference region after administration of a third amount of the contrast agent, the third amount being different from the second amount, preferably larger than the second amount,
   where the trained machine-learning model comprises two submodels: a first submodel and a second submodel, where the first submodel is configured and has been trained to determine, based on the first reference representation and second reference representation of a reference object, at least one model parameter for the second submodel and where the second submodel is configured to generate, based on the first reference representation and/or second reference representation and on the at least one model parameter determined by the first submodel, a synthetic third representation,
providing a first representation, where the first representation represents an examination region of an examination object without contrast agent or after administration of a first amount of the contrast agent,
providing a second representation, where the second representation represents the examination region of the examination object after administration of a second amount of the contrast agent, the second amount being larger than the first amount,
feeding the first representation and the second representation to the trained machine-learning model,
receiving from the trained machine-learning model a synthetic third representation of the examination region of the examination object, where the synthetic third representation represents the reference region after administration of a third amount of the contrast agent, the third amount being different from the second amount, preferably larger than the second amount,
outputting and/or storing the synthetic third representation and/or transmitting the synthetic third representation to a separate computer system.

The present disclosure further provides a contrast agent for use in a radiological examination method, where the radiological examination method comprises the steps of:
providing a trained machine-learning model,
   where the trained machine-learning model has been trained on the basis of training data,
   where the training data for each reference object of a multiplicity of reference objects comprise as target data: (i) a first reference representation of a reference region of the reference object and a second reference representation of the reference region of the reference object as input data and (ii) a third reference representation of the reference region of the reference object,
   where the first reference representation represents the reference region without contrast agent or after administration of a first amount of the contrast agent,
   where the second reference representation represents the reference region after administration of a second amount of the contrast agent, the second amount being larger than the first amount,
   where the third reference representation represents the reference region after administration of a third amount of the contrast agent, the third amount being different from the second amount, preferably larger than the second amount,
   where the trained machine-learning model comprises two submodels: a first submodel and a second submodel, where the first submodel is configured and has been trained to determine, based on the first reference representation and second reference representation of a reference object, at least one model parameter for the second submodel and where the second submodel is configured to generate, based on the first reference representation and/or second reference representation and on the at least one model parameter determined by the first submodel, a synthetic third representation,
providing a first representation, where the first representation represents an examination region of an examination object without contrast agent or after administration of a first amount of the contrast agent,
providing a second representation, where the second representation represents the examination region of the examination object after administration of a second amount of the contrast agent, the second amount being larger than the first amount,
feeding the first representation and the second representation to the trained machine-learning model,
receiving from the trained machine-learning model a synthetic third representation of the examination region of the examination object, where the synthetic third representation represents the reference region after administration of a third amount of the contrast agent, the third amount being different from the second amount, preferably larger than the second amount,
outputting and/or storing the synthetic third representation and/or transmitting the synthetic third representation to a separate computer system.

The present disclosure further provides a kit comprising a computer program product and a contrast agent, wherein the computer program product comprises a computer program that can be loaded into a working memory of a computer system, where it causes the computer system to execute the following steps:
providing a trained machine-learning model,
   where the trained machine-learning model has been trained on the basis of training data,
   where the training data for each reference object of a multiplicity of reference objects comprise as target data: (i) a first reference representation of a reference region of the reference object and a second reference representation of the reference region of the reference object as input data and (ii) a third reference representation of the reference region of the reference object,
   where the first reference representation represents the reference region without contrast agent or after administration of a first amount of the contrast agent,
   where the second reference representation represents the reference region after administration of a second amount of the contrast agent, the second amount being larger than the first amount,
   where the third reference representation represents the reference region after administration of a third amount of the contrast agent, the third amount being different from the second amount, preferably larger than the second amount,
   where the trained machine-learning model comprises two submodels: a first submodel and a second submodel, where the first submodel is configured and has been trained to determine, based on the first reference representation and second reference representation of a reference object, at least one model parameter for the second submodel and where the second submodel is configured to generate, based on the first reference representation and/or second reference representation and on the at least one model parameter determined by the first submodel, a synthetic third representation,
providing a first representation, where the first representation represents an examination region of an examination object without contrast agent or after administration of a first amount of the contrast agent,
providing a second representation, where the second representation represents the examination region of the examination object after administration of a second amount of the contrast agent, the second amount being larger than the first amount,
feeding the first representation and the second representation to the trained machine-learning model,
receiving from the trained machine-learning model a synthetic third representation of the examination region of the examination object, where the synthetic third representation represents the reference region after administration of a third amount of the contrast agent, the third amount being different from the second amount, preferably larger than the second amount,
outputting and/or storing the synthetic third representation and/or transmitting the synthetic third representation to a separate computer system.

### BRIEF DESCRIPTION OF THE DRAWINGS

FIG. 1 shows an embodiment of the second submodel.

FIG. 2 shows in schematic form a further embodiment of the second submodel.

FIG. 3 shows by way of example and in schematic form the training of a machine-learning model for generating a synthetic representation of an examination region of an examination object.

FIG. 4 shows by way of example and in schematic form the generation of a synthetic representation of an examination region of an examination object with the aid of a trained machine-learning model.

FIG. 5 shows by way of example and in schematic form the training of a machine-learning model of the present disclosure.

FIG. 6 shows by way of example and in schematic form the generation of a synthetic representation of an examination region of an examination object with the aid of a trained machine-learning model.

FIG. 7 shows an embodiment for the training of the machine-learning model in the form of a flowchart.

FIG. 8 shows an embodiment for the generation of a synthetic representation of an examination region of an examination object (prediction process) in the form of a flowchart.

FIG. 9 shows by way of example and in schematic form a computer system according to the present disclosure.

FIG. 10 shows by way of example and in schematic form a further embodiment of the computer system.

### DETAILED DESCRIPTION

The subject matters of the present disclosure will be more particularly elucidated below, without distinguishing between the subject matters (method, computer system, computer program (product), use, contrast agent for use, kit). Rather, the elucidations that follow are intended to apply by analogy to all subject matters, irrespective of the context (method, computer system, computer program (product), use, contrast agent for use, kit) in which they occur.

Where steps are stated in an order in the present description or in the claims, this does not necessarily mean that this disclosure is limited to the order stated. Instead, it is conceivable that the steps are also executed in a different order or else in parallel with one another, the exception being when one step builds on another step, thereby making it imperative that the step building on the previous step be executed next (which will however become clear in the individual case). The stated orders thus constitute preferred embodiments.

In certain places the disclosure will be more particularly elucidated with reference to drawings. The drawings show specific embodiments having specific features and combinations of features, which are intended primarily for illustrative purposes; the disclosure is not to be understood as being limited to the features and combinations of features shown in the drawings. Furthermore, statements made in the description of the drawings in relation to features and combinations of features are intended to be generally applicable, that is to say transferable to other embodiments too and not limited to the embodiments shown.

The present disclosure describes means with which, based on a first representation of an examination region of an examination object and on a second representation of the examination region of the examination object, it is possible to predict a third representation of the examination region of the examination object.

Such a predicted third representation is referred to in this disclosure also as a synthetic third representation. The prediction of a third representation is referred to in this disclosure also as the generation of a synthetic third representation.

The term “synthetic” as used herein may mean that the synthetic representation is not the (direct) result of a physical measurement on an actual examination object, but that the image has been generated (calculated) by a machine-learning model. A synonym for the term “synthetic” is the term “artificial”. A synthetic representation may however be based on measured representations, i.e. the machine-learning model is able to generate the synthetic representation on the basis of measured representations.

The “examination object” is normally a living being, preferably a mammal, most preferably a human.

The “examination region” is a part of the examination object, for example an organ or part of an organ or a plurality of organs or another part of the examination object.

For example, the examination region may be a liver, kidney, heart, lung, brain, stomach, bladder, prostate, intestine or a part of said parts or another part of the body of a mammal (for example a human).

In one embodiment, the examination region includes a liver or part of a liver or the examination region is a liver or part of a liver of a mammal, preferably a human.

In a further embodiment, the examination region includes a brain or part of a brain or the examination region is a brain or part of a brain of a mammal, preferably a human.

In a further embodiment, the examination region includes a heart or part of a heart or the examination region is a heart or part of a heart of a mammal, preferably a human.

In a further embodiment, the examination region includes a thorax or part of a thorax or the examination region is a thorax or part of a thorax of a mammal, preferably a human.

In a further embodiment, the examination region includes a stomach or part of a stomach or the examination region is a stomach or part of a stomach of a mammal, preferably a human.

In a further embodiment, the examination region includes a pancreas or part of a pancreas or the examination region is a pancreas or part of a pancreas of a mammal, preferably a human.

In a further embodiment, the examination region includes a kidney or part of a kidney or the examination region is a kidney or part of a kidney of a mammal, preferably a human.

In a further embodiment, the examination region includes one or both lungs or part of a lung of a mammal, preferably a human.

In a further embodiment, the examination region includes a breast or part of a breast or the examination region is a breast or part of a breast of a female mammal, preferably a female human.

In a further embodiment, the examination region includes a prostate or part of a prostate or the examination region is a prostate or part of a prostate of a male mammal, preferably a male human.

The examination region, also referred to as the field of view (FOV), is in particular a volume that is imaged in radiological images. The examination region is typically defined by a radiologist, for example on an overview image. It is also possible for the examination region to be alternatively or additionally defined in an automated manner, for example on the basis of a selected protocol.

The examination region is subjected to a radiological examination.

“Radiology” is the branch of medicine that is concerned with the use of electromagnetic rays and mechanical waves (including for instance ultrasound diagnostics) for diagnostic, therapeutic and/or scientific purposes. Besides X-rays, other ionizing radiation such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present disclosure thus encompasses in particular the following examination methods: computed tomography, magnetic resonance imaging, sonography.

In one embodiment of the present disclosure, the radiological examination is a magnetic resonance imaging examination.

In a further embodiment, the radiological examination is a computed tomography examination.

In a further embodiment, the radiological examination is an ultrasound examination.

The first representation and the second representation are the result of such a radiological examination. The first and the second representation are normally measured radiological images or are generated on the basis of such measured radiological images. The first and the second representation may for example be an MRI image, a CT image and/or an ultrasound image.

The first representation represents the examination region of the examination object without contrast agent or after administration of a first amount of a contrast agent. Preferably, the first representation represents the examination region without contrast agent (native representation).

The second representation represents the examination region of the examination object after administration of a second amount of the contrast agent. The second amount is larger than the first amount (it being possible also for the first amount to be zero, as described).

The expression “after administration of a second amount of the contrast agent” should not be understood as meaning that the first amount and the second amount in the examination region are added together. Thus, the expression “the representation represents the examination region after administration of a (first or second) amount” should rather be understood as meaning: “the representation represents the examination region with a (first or second) amount” or “the representation represents the examination region including a (first or second) amount”. The same applies by analogy to the third amount of the contrast agent too.

The predicted third representation represents the examination region of the examination object after administration of a third amount of the contrast agent. The third amount is different from, preferably larger than, the second amount.

“Contrast agents” are substances or mixtures of substances that improve the depiction of structures and functions of the body in radiological examinations.

In computed tomography, iodine-containing solutions are normally used as contrast agents. In magnetic resonance imaging (MRI), superparamagnetic substances (for example iron oxide nanoparticles, superparamagnetic iron-platinum particles (SIPPs)) or paramagnetic substances (for example gadolinium chelates, manganese chelates, hafnium chelates) are normally used as contrast agents. In the case of sonography, liquids containing gas-filled microbubbles are normally administered intravenously. Examples of contrast agents can be found in the literature (see for example A. S. L. Jascinth et al.: Contrast Agents in computed tomography: A Review, Journal of Applied Dental and Medical Sciences, 2016, vol. 2, issue 2, 143-149; H. Lusic et al.: X-ray-Computed Tomography Contrast Agents, Chem. Rev. 2013, 113, 3, 1641-1666; https://www.radiology.wisc.edu/wp-content/uploads/2017/10/contrast-agents-tutorial.pdf, M. R. Nouh et al.: Radiographic and magnetic resonances contrast agents: Essentials and tips for safe practices, World J Radiol. 2017 Sep. 28; 9(9): 339-349; L. C. Abonyi et al.: Intravascular Contrast Media in Radiography: Historical Development & Review of Risk Factors for Adverse Reactions, South American Journal of Clinical Research, 2016, vol. 3, issue 1, 1-10; ACR Manual on Contrast Media, 2020, ISBN: 978-1-55903-012-0; A. Ignee et al.: Ultrasound contrast agents, Endosc Ultrasound. 2016 November-December; 5(6): 355-362).

MRI contrast agents exert their effect in an MRI examination by altering the relaxation times of structures that take up contrast agents. A distinction can be made between two groups of substances: paramagnetic and superparamagnetic substances. Both groups of substances have unpaired electrons that induce a magnetic field around the individual atoms or molecules. Superparamagnetic contrast agents result in a predominant shortening of T2, whereas paramagnetic contrast agents mainly result in a shortening of T1. The effect of said contrast agents is indirect, since the contrast agent does not itself emit a signal, but instead merely influences the intensity of signals in its vicinity. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem®, Dotagita®, Cyclolux®), gadodiamide (Omniscan®), gadoteridol (ProHance®), gadobutrol (Gadovist®), gadopiclenol (Elucirem, Vueway) and gadoxetic acid (Primovist®/Eovist®).

In one embodiment of the present disclosure, the radiological examination is an MRI examination in which an MRI contrast agent is used.

In a further embodiment, the radiological examination is a CT examination in which a CT contrast agent is used.

In a further embodiment, the radiological examination is a CT examination in which an MRI contrast agent is used.

In one embodiment, both the first amount and the second amount of the contrast agent are smaller than the standard amount.

In a further embodiment, the second amount of the contrast agent corresponds to the standard amount.

In a further embodiment, the first amount of the contrast agent is equal to zero and the second amount of the contrast agent is smaller than the standard amount.

In a further embodiment, the first amount of the contrast agent is equal to zero and the second amount of the contrast agent corresponds to the standard amount.

The standard amount is normally the amount recommended by the manufacturer and/or distributor of the contrast agent and/or approved by a regulatory authority and/or the amount specified in a package leaflet for the contrast agent.

For example, the standard amount of Primovist® is 0.025 mmol Gd-EOB-DTPA disodium/kg body weight.

In one embodiment of the present disclosure, the contrast agent is an agent that includes gadolinium(III) 2-[4,7,10-tris(carboxymethyl)-1,4,7,10-tetrazacyclododec-1-yl]acetic acid (also referred to as gadolinium-DOTA or gadoteric acid).

In a further embodiment, the contrast agent is an agent that includes gadolinium(III) ethoxybenzyldiethylenetriaminepentaacetic acid (Gd-EOB-DTPA); preferably, the contrast agent includes the disodium salt of gadolinium(III) ethoxybenzyldiethylenetriaminepentaacetic acid (also referred to as gadoxetic acid).

In one embodiment of the present disclosure, the contrast agent is an agent that includes gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate (also referred to as gadopiclenol) (see for example WO2007/042504 and WO2020/030618 and/or WO2022/013454).

In one embodiment of the present disclosure, the contrast agent is an agent that includes dihydrogen [(±)-4-carboxy-5,8,11-tris(carboxymethyl)-1-phenyl-2-oxa-5,8,11-triazatridecan-13-oato(5-)]gadolinate(2-) (also referred to as gadobenic acid).

In one embodiment of the present disclosure, the contrast agent is an agent that includes tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate (also referred to as gadoquatrane) (see for example J. Lohrke et al.: Preclinical Profile of Gadoquatrane: A Novel Tetrameric, Macrocyclic High Relaxivity Gadolinium-Based Contrast Agent. Invest Radiol., 2022, 1, 57(10): 629-638; WO2016193190).

In one embodiment of the present disclosure, the contrast agent is an agent that includes a Gd³⁺ complex of a compound of formula (I)

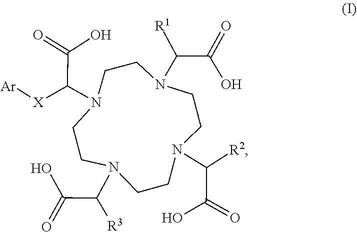

wherein:
Ar is a group selected from:

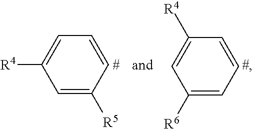

wherein _{#} is a linkage to X,
X is a group selected from:
CH₂, (CH₂)₂, (CH₂)₃, (CH₂)₄ and *—(CH₂)₂—O—CH₂—^{#},
wherein * is a linkage to Ar and ^{#} is a linkage to an acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, a tautomer, a hydrate, a solvate or a salt thereof, or a mixture thereof.

In one embodiment of the present disclosure, the contrast agent is an agent that includes a Gd³⁺ complex of a compound of formula (II)

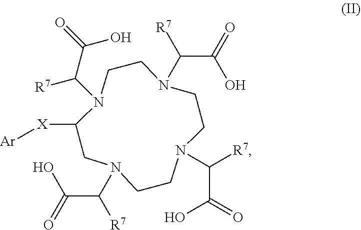

wherein:
Ar is a group selected from:

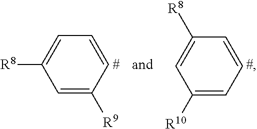

wherein ^{#} is a linkage to X,
X is a group selected from CH₂, (CH₂)₂, (CH₂)₃, (CH₂)₄ and *—(CH₂)₂—O—CH₂—^{#},
wherein * is a linkage to Ar and ^{#} is a linkage to an acetic acid residue,
R⁷ is a hydrogen atom or a group selected from C₁-C₃ alkyl, —CH₂OH, —(CH₂)₂OH and —CH₂OCH₃;
R⁸ is a group selected from: C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are independently a hydrogen atom;
or a stereoisomer, a tautomer, a hydrate, a solvate or a salt thereof, or a mixture thereof.

The term “C₁-C₃ alkyl” denotes a linear or branched, saturated monovalent hydrocarbon group having 1, 2 or 3 carbon atoms, for example methyl, ethyl, n-propyl or isopropyl. The term “C₂-C₄ alkyl” denotes a linear or branched, saturated monovalent hydrocarbon group having 2, 3 or 4 carbon atoms.

The term “C₂-C₄ alkoxy” refers to a linear or branched, saturated monovalent group of the formula (C₂-C₄ alkyl)-O—, in which the term “C₂-C₄ alkyl” is as defined above, for example a methoxy, ethoxy, n-propoxy or isopropoxy group.

In one embodiment of the present disclosure, the contrast agent is an agent that includes gadolinium 2,2′,2″-(10-{1-carboxy-2-[2-(4-ethoxyphenyl)ethoxy]ethyl}-1,4,7,10-tetraazacyclododecane-1,4,7-triyl)triacetate (see for example WO2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 4).

In one embodiment of the present disclosure, the contrast agent is an agent that includes gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate (see for example WO2022/194777, example 15).

In one embodiment of the present disclosure, the contrast agent is an agent that includes gadolinium 2,2′,2″-{10-[(1S)-4-(4-butoxyphenyl)-1-carboxybutyl]-1,4,7,10-tetraazacyclododecane-1,4,7-triyl}triacetate (see for example WO2022/194777, example 31).

In one embodiment of the present disclosure, the contrast agent is an agent that includes gadolinium-2,2′,2″-{(2S)-10-(carboxymethyl)-2-[4-(2-ethoxyethoxy)benzyl]-1,4,7,10-tetraazacyclododecane-1,4,7-triyl}triacetate.

In one embodiment of the present disclosure, the contrast agent is an agent that includes gadolinium 2,2′,2″-[10-(carboxymethyl)-2-(4-ethoxybenzyl)-1,4,7,10-tetraazacyclododecane-1,4,7-triyl]triacetate.

In one embodiment of the present disclosure, the contrast agent is an agent that includes gadolinium(III) 5,8-bis(carboxylatomethyl)-2-[2-(methylamino)-2-oxoethyl]-10-oxo-2,5,8,11-tetraazadodecane-1-carboxylate hydrate (also referred to as gadodiamide).

In one embodiment of the present disclosure, the contrast agent is an agent that includes gadolinium(III) 2-[4-(2-hydroxypropyl)-7,10-bis(2-oxido-2-oxoethyl)-1,4,7,10-tetrazacyclododec-1-yl]acetate (also referred to as gadoteridol).

In one embodiment of the present disclosure, the contrast agent is an agent that includes gadolinium(III) 2,2′,2″-(10-((2R,3S)-1,3,4-trihydroxybutan-2-yl)-1,4,7,10-tetraazacyclododecane-1,4,7-triyl)triacetate (also referred to as gadobutrol or Gd-DO3A-butrol).

A representation of an examination region for the purposes of the present disclosure is preferably a radiological image of the examination region.

A representation of an examination region (or a reference representation of a reference region) may for the purposes of the present disclosure be a representation in real space (image space), a representation in frequency space, a representation in the projection space or a representation in another space.

In a representation in real space, also referred to in this description as real-space depiction or real-space representation, the examination region is normally represented by a large number of image elements (for example pixels or voxels or doxels), which may for example be in a raster arrangement in which each image element represents a part of the examination region, wherein each image element may be assigned a colour value or grey value. The colour value or grey value represents a signal intensity, for example the attenuation of X-rays. A format widely used in radiology for storing and processing representations in real space is the DICOM format. DICOM (Digital Imaging and Communications in Medicine) is an open standard for storing and exchanging information in medical image data management.

In a representation in frequency space, also referred to in this description as frequency-space depiction or frequency-space representation, the examination region is represented by a superposition of fundamental vibrations. For example, the examination region may be represented by a sum of sine and cosine functions having different amplitudes, frequencies and phases. The amplitudes and phases may be plotted as a function of the frequencies, for example, in a two- or three-dimensional plot. Normally, the lowest frequency (origin) is placed in the centre. The further away from this centre, the higher the frequencies. Each frequency can be assigned an amplitude representing the frequency in the frequency-space depiction and a phase indicating the extent to which the respective vibration is shifted towards a sine or cosine vibration.

A representation in real space can for example be converted (transformed) by a Fourier transform into a representation in frequency space. Conversely, a representation in frequency space can for example be converted (transformed) by an inverse Fourier transform into a representation in real space.

Details about real-space depictions and frequency-space depictions and their respective interconversion are described in numerous publications, see for example https://see.stanford.edu/materials/lsoftaee261/book-fall-07.pdf.

A representation of an examination region in the projection space is normally the result of a computed tomography examination prior to image reconstruction. In other words: the raw data obtained in the computed tomography examination can be understood as a projection-space depiction. In computed tomography, the intensity or attenuation of X-radiation as it passes through the examination object is measured. From this, projection values can be calculated. In a second step, the object information encoded by the projection is transformed into an image (real-space depiction) through a computer-aided reconstruction. The reconstruction can be effected with the Radon transform. The Radon transform describes the link between the unknown examination object and its associated projections.

Details about the transformation of projection data into a real-space representation are described in numerous publications, see for example K. Catch: The Radon Transformation and Its Application in Tomography, Journal of Physics Conference Series 1903(1):012066.

A representation of the examination region can also be a representation in the Hough space. For the recognition of geometric objects in an image, edge detection is followed by the creation, by what is known as a Hough transform, of a dual space in which all possible parameters of the geometric object are entered for each point in the image lying at an edge. Each point in dual space accordingly corresponds to a geometric object in image space. For a straight line this can be for example the slope and the y-axis section of the straight line and for a circle it can be the centre point and radius of the circle. Details about the Hough transform can be found in the literature (see for example A. S. Hassanein et al.: A Survey on Hough Transform, Theory, Techniques and Applications, arXiv:1502.02160v1).

There are further spaces in which it is possible for there to be representations of the examination region. For the sake of simplicity and clarity, the disclosure is in large parts of the description described on the basis of real-space representations. This should not however be understood as limiting. Those skilled in the art of image analysis know how to apply the appropriate parts of the description to representations other than real-space representations.

The generation of the synthetic third representation (i.e. the prediction of the third representation), is effected with the aid of a trained machine-learning model.

A “machine learning model” can be understood as meaning a computer-implemented data processing architecture. Such a model is able to receive input data and to supply output data on the basis of said input data and model parameters. Such a model is able to learn a relationship between the input data and the output data through training. During training, the model parameters can be adjusted so as to supply a desired output for a particular input.

During the training of such a model, the model is presented with training data from which it can learn. The trained machine-learning model is the result of the training process. Besides input data, the training data includes the correct output data (target data) that the model is intended to generate on the basis of the input data. During training, patterns that map the input data onto the target data are identified.

In the training process, the input data of the training data are input into the model, and the model generates output data. The output data are compared with the target data. Model parameters are altered so as to reduce the differences between the output data and the target data to a (defined) minimum. The modification of model parameters in order to reduce the differences can be done using an optimization process such as a gradient process.

The differences can be quantified with the aid of a loss function. A loss function of this kind can be used to calculate a loss for a given set of output data and target data. The aim of the training process may consist of altering (adjusting) the parameters of the machine-learning model so as to reduce the loss for all pairs of the training data set to a defined) minimum.

For example, if the input data and the target data are numerical values, the loss function can be the absolute difference between these values. In this case, a high absolute loss value can mean that one or more model parameters needs to be changed to a substantial degree.

For example, for output data in the form of vectors, difference metrics between vectors such as the mean square error, a cosine distance, a norm of the difference vector such as a Euclidean distance, a Chebyshev distance, an Lp norm of a difference vector, a weighted norm or another type of difference metric of two vectors can be chosen as the loss function.

In the case of higher-dimensional outputs, such as two-dimensional, three-dimensional or higher-dimensional outputs, an element-by-element difference metric can for example be used. Alternatively or in addition, the output data may be transformed into for example a one-dimensional vector before calculation of a loss value.

The model described herein is understood as a “machine-learning model”, since it includes at least one component that can be trained, for example, in a monitored learning process. Components of the model described herein are also referred to in this description as submodels. Such submodels may be employed independently of one another and/or be interconnected in the (overall) model such that the output of one submodel is fed directly to the submodel that follows. In other words: submodels may be externally recognizable as separate entities and/or be interconnected such that they are perceived externally as a single model.

The trained machine-learning model used to generate a synthetic third representation comprises at least two submodels: a first submodel and a second submodel. In addition to the first submodel and second submodel, the trained machine-learning model may comprise one or more further submodels.

The first submodel is a machine-learning model. A training process trains the first submodel on the basis of training data. The first submodel is configured and trained to determine (predict) at least one model parameter for the second submodel.

The second submodel is a mechanistic (deterministic) model. Mechanistic models are based on fundamental principles and known relationships within a system. They are often derived from scientific theories and field-specific knowledge. Mechanistic models describe the underlying mechanisms of a system with the aid of mathematical equations or physical laws. They aim to simulate the behaviour of the system based on an understanding of its components and interactions.

Machine-learning models, on the other hand, are data-driven and learn patterns and relationships from input data without the relationships being explicitly programmed.

Thus, whereas mechanistic models are based on fundamental principles and aim to represent the underlying system mechanisms, machine-learning models learn patterns and relationships directly from data without explicitly programming these relationships.

The mechanistic model is thus based on physical laws. In radiological examinations, a signal produced by a contrast agent is normally dependent on the amount (e.g. concentration) of the contrast agent in the examination region. For example, the signal strength may over a defined concentration range show a linear dependence or another form of dependence on the concentration of the contrast agent in the examination region. The functional dependence of the signal strength on the concentration can be utilized to create a mechanistic model.

The mechanistic model includes at least one model parameter that, in part at least, also determines the signal intensity distribution in the synthetic third representation. The at least one model parameter may for example represent the dependence of the signal strength on the concentration of the contrast agent in the examination region. The at least one model parameter may also comprise one or more parameters of a filter that is applied to a representation of the examination region.

This is elucidated in more detail below with reference to two examples, but without any intention to restrict the disclosure to these examples.

If a first real-space representation of an examination region of an examination object that represents the examination region without contrast agent (native representation) is subtracted from a second real-space representation of the examination region of the examination object that represents the examination region after administration of a second amount of contrast agent that is different from zero, the result will be a real-space representation of the examination region in which the signal intensity distribution is determined solely by the contrast agent (representation of the contrast-agent distribution). In such a subtraction it is normally the colour values or grey values of corresponding image elements that are subtracted from one another. Corresponding image elements are those image elements that represent the same subregion of the examination region.

If this representation of the contrast-agent distribution is added to the first (native) real-space representation of the examination region, this in turn gives rise to the second real-space representation of the examination region. In such an addition too it is normally the colour values or grey values of corresponding image elements that are added.

If the representation of the contrast-agent distribution is added only in part (for example after being multiplied by a factor of 0.5 or by another factor between 0 and 1) to the first (native) real-space representation of the examination region, a real-space representation of the examination region is obtained in which the signal intensity distribution produced by the contrast agent is larger than in the first (native) real-space representation and smaller than in the second real-space representation.

If the representation of the contrast-agent distribution is multiplied by a factor greater than 1 and the result added to the first (native) real-space representation of the examination region, a real-space representation of the examination region is obtained (optionally after normalization) in which the signal intensity distribution produced by the contrast agent is greater than in the second real-space representation. Such a multiplication is normally carried out, as in the case of the above-described subtraction and addition, by multiplying the colour values or grey values of all image elements by the factor.

In other words: the representation of the contrast-agent distribution can, after being multiplied by a gain factor α, be added to the first or second representation in order to enhance (or to reduce) relative to other subregions without contrast agent the contrast enhancement of subregions of the examination region that contain contrast agent. The signal intensities of subregions that contain more contrast agent than other subregions can be enhanced relative to the signal intensities of these other subregions.

Negative α values are also possible, which can for example be chosen so that regions of the examination region that experience a contrast agent-induced signal enhancement in the representation generated by measurement are completely dark (black) in the synthetic third representation.

The gain factor α is thus a positive or negative real number that can be varied with the contrast enhancement; by varying the gain factor α it is thus possible to vary the contrast between regions with contrast agent and regions without contrast agent.

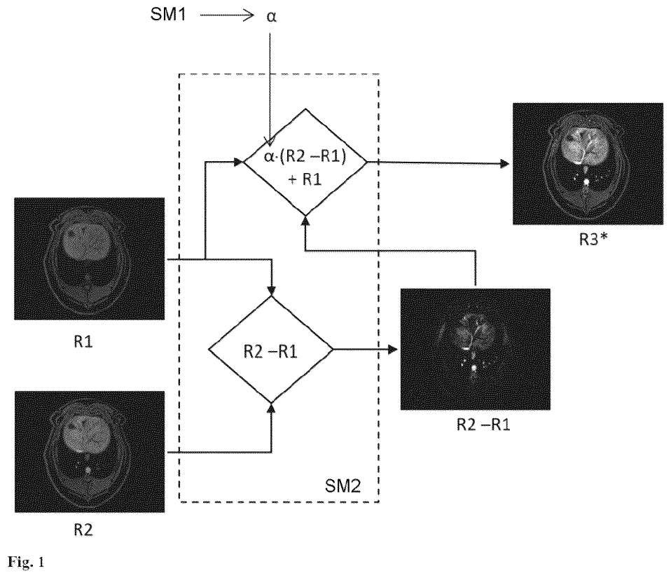

FIG. 1 shows an embodiment of the second submodel. The second submodel SM2 is configured to generate, based on a first representation R1 of an examination region of an examination object, on a second representation R2 of the examination region of the examination object and on the gain factor α as model parameter, a synthetic third representation R3* of the examination region of the examination object.

The examination object is in the present example a pig and the examination region includes the pig's liver.

The first representation R1 is a magnetic resonance image that represents the examination region in real space without contrast agent.

The second representation R2 represents the same examination region of the same examination object as the first representation R1 in real space. The second representation R2 is likewise a magnetic resonance image.

The second representation R2 represents the examination region after administration of a second amount of a contrast agent. In the present example, an amount of 25 μmol per kg body weight of a hepatobiliary contrast agent was administered intravenously to the examination object. The second representation R2 represents the examination region in the so-called arterial phase (see for example DOI:10.1002/jmri.22200).

A hepatobiliary contrast agent has the characteristic features of being specifically taken up by liver cells (hepatocytes), accumulating in the functional tissue (parenchyma) and enhancing contrast in healthy liver tissue. An example of a hepatobiliary contrast agent is the disodium salt of gadoxetic acid (Gd-EOB-DTPA disodium), which is described in U.S. Pat. No. 6,039,931A and is commercially available under the trade names Primovist® and Eovist®. Further hepatobiliary contrast agents are described inter alia in WO 2022/194777.

The first representation R1 and the second representation R2 are fed to the second submodel SM2. On the basis of the first representation R1 and the second representation R2, the second submodel SM2 generates a synthetic third representation R3*. Synthetic representations are indicated by a * in this disclosure.

In the example shown in FIG. 1, the generation of the synthetic third representation R3* involves subtracting the first representation R1 from the second representation R2. The difference R2−R1 of the first representation R1 from the second representation R2 is a representation of the contrast-agent distribution, as described above. The generation of the synthetic third representation R3* additionally involves adding the difference R2−R1 of the first representation from the second representation to the first representation a times (R3*=α·(R2−R1)+R1). The gain factor α is provided by the first submodel SM1.

If negative grey/colour values occur when subtracting the first representation R1 from the second representation R2, these negative values can be set to zero (or another value) to avoid negative values.

The difference (R2−R1) represents the contrast enhancement (signal intensity distribution) produced in the examination region by the second amount of contrast agent.

The difference (R2−R1) is multiplied by the gain factor α and the multiplication result added to the first representation R1. This generates the synthetic third representation R3*. In the example shown in FIG. 1, the gain factor α=3, i.e. the difference (R2−R1) is added to the first representation R1 three times.

The third representation R3* can be subjected to a normalization, that is to say the grey/colour values can be multiplied by a factor such that the grey/colour value having the highest value is represented for example by the grey tone/hue “white” and the grey/colour value having the lowest value is represented for example by the grey/colour tone “black”.

The mechanistic model described in relation to FIG. 1 is based on the assumption that the signal intensity represented by grey values or colour values in a representation of the examination region shows linear dependence on the amount of contrast agent administered. This is the case particularly in many MRI examinations. The linear dependence allows the contrast to be varied by varying the gain factor α. A gain factor of α=2 thus means that the third amount of contrast agent is equal to twice the second amount.

It should be noted that instead of linear dependence, the mechanistic model can also be based on another dependence. The dependence can be determined empirically.

In the example shown in FIG. 1, the second submodel SM2 thus consists of mathematical operations that execute the subtractions, multiplications and additions on the basis of the grey/colour values of the individual image elements (for example pixels, voxels).

The above-described operations for generating a synthetic third representation with variable contrast enhancement can be carried out in an analogous manner also in other spaces on the basis of representations other than real-space representations, for example in frequency space on the basis of frequency-space representations.

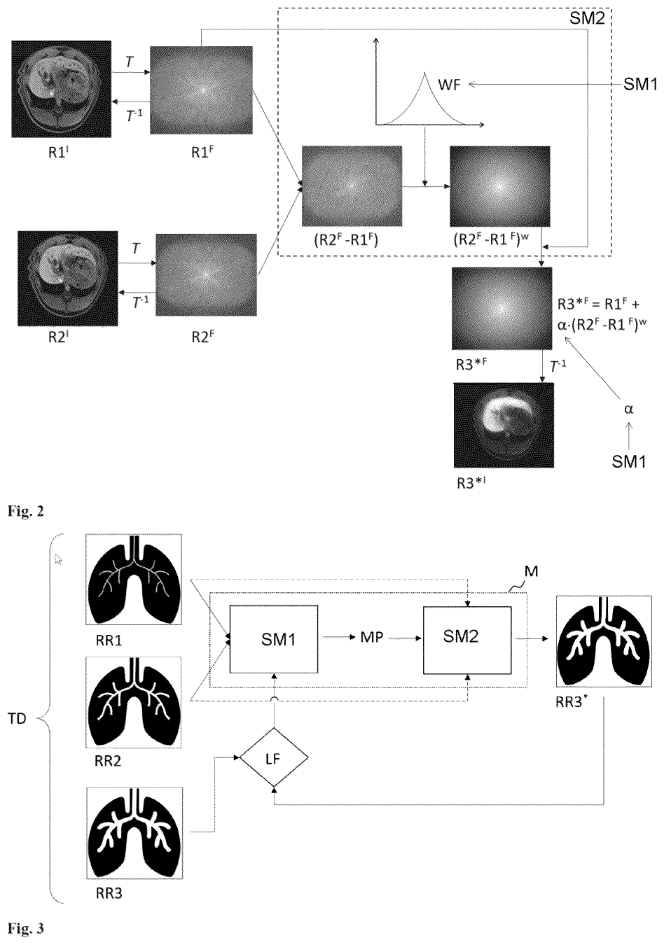

FIG. 2 shows in schematic form a further embodiment of a second submodel.

The second submodel SM2 depicted in FIG. 2 is configured to generate, based on a first representation R1^{F} of an examination region of an examination object in frequency space and on a second representation R2^{F} of the examination region of the examination object in frequency space, a synthetic third representation R3*^{F} of the examination region of the examination object in frequency space.

The representations R1^{F} and R2^{F} of the examination region of the examination object in frequency space can be obtained for example from the corresponding real-space representations R1^{I} and R2^{I}.

The first representation R1^{I} represents the examination region in real space without contrast agent or after administration of a first amount of a contrast agent. The examination region shown in FIG. 2 includes a liver of a pig. The first representation R1^{I} is a magnetic resonance image.

The first real-space representation R1^{I} can be converted into the first representation R1^{F} of the examination region in frequency space through a transform operation T, for example a Fourier transform. The first frequency-space representation R1^{F} represents the same examination region of the same examination object as the first real-space representation R1^{I}, likewise without contrast agent or after administration of the first amount of the contrast agent.

The first frequency-space representation R1^{F} can be converted into the first real-space representation R1^{I} by means of a transform operation T⁻¹, for example through an inverse Fourier transform. The transform operation T⁻¹ is the inverse transform of transform operation T.

The second representation R2^{I} represents the same examination region of the same examination object as the first representation R1^{I} in real space. The second real-space representation R2^{I} represents the examination region after administration of a second amount of the contrast agent. The second amount is larger than the first amount (it being possible also for the first amount to be zero, as described). The second representation R2^{I} is likewise a magnetic resonance image. As contrast agent, the disodium salt of gadoxetic acid (Gd-EOB-DTPA disodium) was in the example shown in FIG. 2 used as a hepatobiliary MRI contrast agent.

The second real-space representation R2^{I} can be converted into the second representation R2^{F} of the examination region in frequency space through the transform operation T. The second frequency-space representation R2^{F} represents the same examination region of the same examination object as the second real-space representation R2^{I}, likewise after administration of the second amount of contrast agent.

The second frequency-space representation R2^{F} can be converted into the second real-space representation R2^{I} by means of the transform operation T⁻¹.

In the example shown in FIG. 2, the first frequency-space representation R1^{F} and the second frequency-space representation R2^{F} are fed to the second submodel SM2. On the basis of the first frequency-space representation R1^{F} and the second frequency-space representation R2^{F}, the second submodel SM2 generates the synthetic third frequency-space representation R3*^{F}. The synthetic third frequency-space representation R3*^{F} can be converted into a synthetic third real-space representation R3*^{I} through a transform operation T⁻¹ (for example an inverse Fourier transform).

The second submodel SM2 shown in FIG. 2 does not include the transform operation T that converts the first real-space representation R1^{I} into the first frequency-space representation R1^{F} and converts the second real-space representation R2^{I} into the second frequency-space representation R2^{F}. Likewise, the second submodel SM2 shown in FIG. 2 does not include the transform operation T⁻¹ that converts the synthetic third frequency-space representation R3*^{F} into the synthetic third real-space representation R3*^{I}. However, it is conceivable that the transform operation T and/or transform operation T⁻¹ are component(s) of the second submodel SM2, i.e. it is conceivable that the second submodel SM2 executes the transform operation T and/or operation T⁻¹.

The second submodel SM2 subtracts the first frequency-space representation R1^{F} from the second frequency-space representation R2^{F} (R2^{F}−R1^{F}). The result is a representation of the signal intensity distribution in frequency space produced by the contrast agent in the examination region.

The difference R2^{F}−R1^{F} is multiplied by a weight function WF that weights low frequencies more highly than high frequencies. In this case, the amplitudes of the fundamental vibrations are multiplied by a weight factor that increases as the frequencies become smaller. This step is an optional step that can be executed to increase the signal-to-noise ratio in the synthetic third representation, especially at higher values for the gain factor α (for example values greater than 3, 4, or 5). The result of this frequency-dependent weighting is the weighted representation (R2^{F}−R1^{F})^{W}.

Contrast information is represented in a frequency-space depiction by low frequencies, while the higher frequencies represent information about fine structures. Such weighting thus means that a higher weighting will be given to frequencies making a higher contribution to contrast than to those making a smaller contribution. Image noise is typically evenly distributed in the frequency depiction. The frequency-dependent weight function has the effect of a filter. The filter increases the signal-to-noise ratio by reducing the spectral noise density for high frequencies.

Preferred weight functions are Hann function (also referred to as the Hann window) and Poisson function (Poisson window).

Examples of other weight functions can be found for example at https://de.wikipedia.org/wiki/Fensterfunktion #Beispiele_von_Fensterfunktionen; F. J. Harris et al.: On the Use of Windows for Harmonic Analysis with the Discrete Fourier Transform, Proceedings of the IEEE, vol. 66, No. 1, 1978; https://docs.scipy.org/doc/scipy/reference/signal.windows.html; K. M. M Prabhu: Window Functions and Their Applications in Signal Processing, CRC Press, 2014, 978-1-4665-1583-3).

The weighted difference (R2^{F}−R1^{F})^{W} is in a next step multiplied by a gain factor α and added to the first frequency-space representation R1^{F}. The result is a synthetic third representation R3*^{F}=R1^{F}+α·(R2^{F}−R1^{F})^{W} of the examination region of the examination object in frequency space. In a further step, the synthetic third frequency-space representation R3*^{F} is converted into the synthetic third representation R3*¹ of the examination region of the examination object in real space through the transform operation T⁻¹ (for example an inverse Fourier transform).

The synthetic third representation R3*^{I} represents the examination region of the examination object after administration of a third amount of the contrast agent. The third amount depends on the gain factor α. For example, if the gain factor is 3 and if the signal intensity distribution represented by the grey/colour values shows linear dependence on the amount of contrast agent, then the third amount corresponds to three times the difference of the first amount from the second amount.

If a second submodel SM2 as depicted in FIG. 1 is used to generate the third representation, then not only is the contrast enhanced by a gain factor greater than 1 (α>1), but the noise too is enhanced to the same extent. A second submodel SM2 as depicted in FIG. 2 is able to achieve a certain reduction in noise through weighting with the weight function in frequency space.

The gain factor α may be a model parameter of the second submodel that is provided (determined, predicted) by the first submodel.

Likewise, one or more parameters of the weight function may be model parameters of the second submodel that are provided (determined, predicted) by the first submodel.

If the weight function is for example a two-dimensional Gaussian function, then it has the formula

$$w f ( x , y ) = \frac{1}{2 π {σ}^{2}} {e}^{- \frac{{x}^{2} + {y}^{2}}{2 {σ}^{2}}}$$

In this formula, wf is the frequency-dependent weight factor by which the amplitudes of the fundamental vibrations of the frequency-space representation R2^{F}−R1^{F} are multiplied. x are the frequencies along the horizontal axis and y are the frequencies along the vertical axis. π is the number pi and σ is the standard deviation. The standard deviation σ may be a model parameter of the second submodel that is determined (provided) by the first submodel.

For other weight functions such as the Hann function (Hann window) and Poisson function (Poisson window), it is also by analogy possible for parameters thereof that characterize the respective weight function to be model parameters of the second submodel that are determined (provided) by the first submodel.

The at least one model parameter determined by the first submodel may for example be one or more parameters of the frequency-dependent weight function that determine the weight factor by which the amplitudes of the individual frequencies of the fundamental vibrations are multiplied. The at least one model parameter determined by the first submodel may for example comprise at least one parameter that determines the width of the weight function (window function), the slope by which the weight function falls as the frequency increases and/or other properties of the weight function.

It is also conceivable that a model parameter determines which weight function is used by the second submodel to carry out a frequency-dependent filtering. It is conceivable that, during the training process, various weight functions are “tested” and the machine-learning model is trained to select the weight function that results in the best-possible prediction of the third representation.

The first submodel can be trained to determine at least one model parameter (for example the gain factor α, parameters of a weight function and/or further/other model parameters) that results in a synthetic third representation having properties that are determined by target data. The target data thus do not themselves need to include the at least one model parameter. The target data may comprise a (measured) third representation. The first model can be trained to choose the at least one model parameter such that the synthetic third representation approximates as closely as possible to the (measured) third representation (ground truth).

This is shown by way of example and in schematic form in FIG. 3.

FIG. 3 shows by way of example and in schematic form the training of a machine-learning model for generating a synthetic representation of an examination region of an examination object.

The training takes place on the basis of training data TD. The training data TD comprise as target data for each reference object of a multiplicity of reference objects: (i) a first reference representation RR1 of a reference region of the reference object and a second reference representation RR2 of the reference region of the reference object as input data and (ii) a third reference representation RR3 of the reference region of the reference object, FIG. 1 shows a single data set for a reference object. The reference object is a human and the reference region includes the lung of the human.

The term “reference” is used in this description to distinguish the phase of training the machine-learning model from the phase of using the trained model for the generation of a synthetic representation. The term “reference” otherwise has no limitation on meaning. A “reference object” is an object, the data of which (for example reference representations) are used to train the machine-learning model. On the other hand, data of an examination object are utilized in order to use the trained model for prediction. The term “(reference) representation” as used herein may mean that the corresponding statement applies both to a representation of an examination object and to a reference representation of a reference object. All other statements made in this description in relation to an examination object similarly apply to each reference object too and vice versa. Each reference object is, like the examination object, normally a living being, preferably a mammal, most preferably a human. The “reference region” is a part of the reference object. The reference region is normally (but not necessarily) the examination region of the examination object. In other words, when the examination region is an organ or part of an organ (for example the liver or part of the liver) of the examination object, the reference region of each such reference object is preferably the corresponding organ or corresponding part of the organ of the respective reference object. All other statements made in this description in relation to an examination region similarly apply to the reference region too and vice versa.

The first reference representation RR1, the second reference representation RR2 and the third reference representation RR3 are radiological images; they may for example be MRI images and/or CT images and/or X-ray images.

The first reference representation RR1 represents the reference region of the reference object without contrast agent or after administration of a first amount of a contrast agent. The second reference representation RR2 represents the reference region of the reference object after administration of a second amount of a contrast agent. The second amount is larger than the first amount (it being possible also for the first amount to be zero, as described). The third reference representation RR3 represents the reference region of the reference object after administration of a third amount of a contrast agent. The third amount is different from the second amount, preferably the third amount is larger than the second amount.

For example, if the second amount is smaller than the standard amount, the third amount may be equal to the standard amount. However, it is also possible for the third amount to be larger than the standard amount.

The machine-learning model M comprises two submodels, SM1 and SM2. The first submodel SM1 is configured and trained to determine (predict), based on the first reference representation RR1 and the second reference representation RR2 (and on the basis of model parameters of the first submodel SM1), at least one model parameter MP for the second submodel SM2. The second submodel SM2 is configured to generate, based on the first reference representation RR1 and/or the second reference representation RR2, a synthetic third representation RR3*.

The first submodel SM1 may for example be an artificial neural network (as more particularly described hereinbelow) or include such a network. The second submodel SM2 may be a mechanistic model as described in relation to FIG. 1 or FIG. 2 or another mechanistic model. The second submodel SM2 may be a model disclosed in EP 22207079.9, EP 22207080.7 and/or EP 23168725.2.

The first reference representation RR1 and second reference representation RR2 are fed to the first submodel SM1 and the first submodel SM1 supplies the at least one model parameter MP to the second submodel. The at least one model parameter MP may for example be or include the gain factor α. The at least one model parameter MP may be one or more parameters of a weight function and/or of a filter function or include one or more such parameters.

It is possible to co-register the first reference representation RR1 and the second reference representation RR2 before feeding them to the first submodel SM1. “Co-registration” (also known in the prior art as “image registration”) is employed to bring two or more real-space depictions of the same examination region into the best possible conformity with one other. One of the real-space depictions is defined as the reference image, the other is termed the object image. In order to optimally fit this to the reference image, a compensating transform is calculated.

It is also possible to co-register representations in frequency space; it should here be noted that a translation in real space constitutes an additive linear phase ramp in frequency space. Scaling and rotation are on the other hand retained in the Fourier and inverse Fourier transform—scaling and rotation in frequency space is also scaling and rotation in real space (see for example S. Skare: Rigid Body Image Realignment in Image Space vs. k-Space, ISMRM Scientific Workshop on Motion Correction, 2014, https://cds.ismrm.org/protected/Motion_14/Program/Syllabus/Skare.pdf).

The second submodel SM2 accepts the at least one model parameter and generates the synthetic third reference representation RR3*. The synthetic third reference representation RR3* can be compared with the third reference representation RR3 of the target data. In the example shown in FIG. 3, a loss function LF is used to quantify differences between the synthetic third reference representation RR3* and the third reference representation RR3. The differences may be used to modify model parameters of the first submodel (SM1) in an optimization process (for example a gradient process) so as to minimize the differences.

The described process is carried out one or more times for a multiplicity of reference representations of a multiplicity of reference objects. The training can be ended when the calculated loss determined by the loss function attains a predefined minimum value and/or the loss value cannot be reduced further by modifying model parameters.

From FIG. 3 it is clear that, to train the machine-learning model M, the at least one model parameter MP to be determined by the first submodel SM1 does not need to be known. In other words, the at least one model parameter MP does not need to be a component of the training data/target data. Instead, the training data include the (measured) third reference representation RR3 as target data (ground truth). The first submodel SM1 suggests at least one model parameter MP, on the basis of which the second submodel SM2 generates the synthetic third reference representation RR3*. If differences occur between the synthetic third reference representation RR3* and the (measured) third reference representation RR3, these are detected and quantified by means of the loss function LF. Model parameters of the first submodel SM1 are modified so as to reduce/minimize the differences.

From FIG. 3 it is also clear that the second submodel SM2 does not itself undergo training. It is only the first submodel SM1 that is trained. If there are differences between the synthetic third reference representation RR3* and the (measured) third reference representation RR3, it is only model parameters of the first submodel SM1 that undergo modification in an optimization process (for example a gradient process) to reduce the differences. Modifying the model parameters of the first submodel SM1 normally results also in a change to the at least one model parameter MP determined by the first submodel SM1. The at least one model parameter MP is however the result of an adjustment of the model parameters of the first submodel SM1 during training and is not a training measure.

The second submodel SM2 can nevertheless be included in the training: if the second submodel SM2 is differentiable, then the machine-learning model M can undergo end-to-end training.

The advantage of dividing the machine-learning model into at least two submodels is that the second submodel is based on a mechanistic approach to the generation of the synthetic third representation and accordingly supplies trackable results. The generation of the synthetic third representation stays within the limits specified by the second submodel. It is not possible for a synthetic third representation to be generated that is not in conformity with the mechanistic model. The number of model parameters modified to achieve a possible loss-free fit between the synthetic third representation and the (measured) third representation is only small compared with an artificial neural network having a large number of nodes and layers. Once it has been trained, the first submodel determines the model parameters for the second submodel that result in optimal prediction, i.e. a prediction with which the synthetic representation achieves an optimal approximation of the measured representation.

The at least one model parameter determined by the first submodel can be outputted (for example displayed on a monitor and/or printed on a printer) so that a user is able to check the at least one determined model parameter. The user is thus able to check whether the at least one model parameter determined by the first submodel is within expected limits and is thus meaningful.

Such a check whether the at least one model parameter determined by the first submodel is within predefined limits can also take place in an automated manner. “Automated” may refer to without human assistance. The at least one model parameter determined by the first submodel can be compared with one or more predefined limit values. If the at least one model parameter is above a predefined upper limit value or below a predefined lower limit value, an output can be issued stating that the at least one model parameter determined by the first submodel is outside a define range and that the synthetic third representation may accordingly be erroneous.

One or more limit values may be set (predefined) for example on the basis of physical laws and/or statistical calculations and/or empirically. When, for example, the first reference representation represents the examination region without contrast agent, the second reference representation represents the examination region with a second amount of contrast agent, the third reference representation represents the examination region with a third amount of contrast agent and the third amount is larger than the second amount, then the gain factor α described previously must be greater than 1. If the at least one model parameter determined by the first submodel includes such a gain factor and this factor is less than 1, this means that the second submodel is not staying within physical laws and that the synthetic third reference representation generated by the second submodel may be erroneous.

As described in this disclosure, the first submodel is configured and has been trained to determine, based on a first (reference) representation and a second (reference) representation of an examination region (or reference region) of an examination object (or reference object), at least one model parameter for the second submodel. In this context, the term “based on” may refer to that the first (reference) representation and the second (reference) representation are input into the first submodel as input data and the first submodel provides (e.g. outputs) the at least one model parameter in response to this input, such that the second submodel is able to use said at least one model parameter.

The second submodel is configured to generate, based on the first (reference) representation and/or the second (reference) representation and the at least one model parameter determined by the first submodel, a synthetic third reference representation. This means that the first (reference) representation and/or the second (reference) representation are input into the second submodel as input data and the second submodel generates and provides (e.g. outputs) the synthetic third (reference) representation in response to this input. The at least one model parameter determined by the first submodel is here a model parameter of the second submodel that influences how the synthetic third (reference) representation is generated.

The first submodel may be an artificial neural network or include such a network.

An “artificial neural network” comprises at least three layers of processing elements: a first layer having input neurons (nodes), an N-th layer having at least one output neuron (node) and N−2 inner layers, where N is a natural number and greater than 2.

The input neurons serve to receive the first and second (reference) representations. There may for example be one input neuron for each pixel or voxel of a (reference) representation when the representation is a real-space depiction in the form of a raster graphic, or one input neuron for each frequency present in the (reference) representation when the representation is a frequency-space depiction. There may be additional input neurons for additional input data (for example information about the examination region/reference region, about the examination object/reference object, about the conditions prevailing during the generation of the input representation, information about the state that the (reference) representation represents, and/or information about the time or time interval at/during which the (reference) representation had been generated).

The output neurons serve to output the at least one model parameter for the second submodel.

The processing elements of the layers between the input neurons and the output neurons are connected to one another in a predetermined pattern with predetermined connection weights.

The artificial neural network may be a convolutional neural network (CNN for short) or include such a network.

A convolutional neural network is capable of processing input data in the form of a matrix. This makes it possible to use as input data digital radiological images depicted in the form of a matrix (e.g. width×height×colour channels). A normal neural network, for example in the form of a multilayer perceptron (MLP), requires on the other hand a vector as input, i.e. in order to use a radiological image as input, the pixels or voxels of the radiological image would have to be rolled out in a long chain one after the other. This means that normal neural networks are for example not able to recognize objects in a radiological image independently of the position of the object in the image. The same object at a different position in the image would have a completely different input vector.

A CNN normally consists essentially of an alternately repeating array of filters (convolutional layer) and aggregation layers (pooling layer) terminating in one or more layers of fully connected neurons (dense/fully connected layer).

The first submodel can for example have an architecture based on the architecture depicted in FIG. 5 of WO 2019/074938 A1: input layers for the first and the second (reference) representation followed by a series of encoder layers may be used to compress the (reference) representations and the information contained therein in a feature vector. The encoder layers may be followed by layers of fully connected neurons (fully connected layers), which are followed lastly by an output layer. The layers of fully connected neurons can calculate the at least one model parameter from the feature vector, for example in the form of a regression. The output layer can have as many output neurons as there are model parameters determined (calculated) by the first submodel for the second submodel.

FIG. 3 shows a training process based on reference representations in real space. It is likewise possible for the training to be carried out partly or entirely on the basis of reference representations in frequency space.

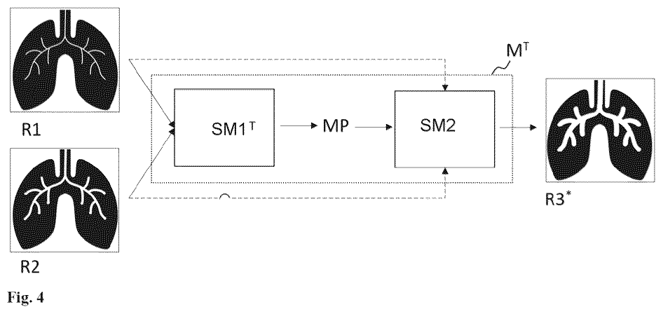

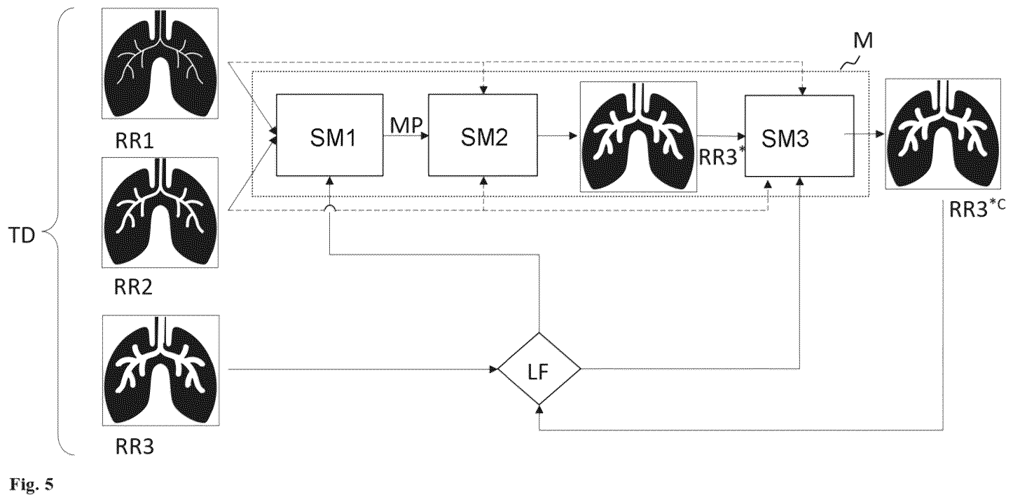

Once it has been trained, the machine-learning model of the present disclosure can be used for prediction. This is shown by way of example and in schematic form in FIG. 4. FIG. 4 shows a prediction process based on representations in real space. It is likewise possible for prediction to be carried out partly or entirely on the basis of representations in frequency space, particularly when the corresponding training process has also been carried out partly or entirely on the basis of reference representations in frequency space.

FIG. 4 shows by way of example and in schematic form the generation of a synthetic representation of an examination region of an examination object with the aid of a trained machine-learning model.

The trained machine-learning model M^{T} may for example have been trained as described in relation to FIG. 3. The superscripted T in the reference sign M^{T} for the machine-learning model M^{T} serves to indicate that this is a trained model. The same applies also to the first submodel SM1^{T}.

The trained machine-learning model M^{T} comprises a trained first submodel SM1^{T} and a second submodel SM2. The trained first submodel SM1^{T} may be an artificial neural network or include such a network. The second submodel SM2 may be a mechanistic model as described in relation to FIG. 1 or FIG. 2, or another model. The second submodel SM2 may be a model disclosed in EP 22207079.9, EP 22207080.7 and/or EP 23168725.2.

In a first step, a first representation R1 of an examination region of an examination object and a second representation R2 of the examination region of the examination object are received.

The term “receiving” encompasses both the retrieving of representations and the accepting of representations transmitted for example to the computer system of the present disclosure. The representations may be received from a computed tomography system, from a magnetic resonance imaging system or from an ultrasound scanner. The representations may be read from one or more data storage media and/or transmitted from a separate computer system.

The first representation R1 and the second representation R2 are fed to the trained first submodel SM1^{T}.

It is possible to co-register the first representation R1 and the second representation R2 before feeding them to the first submodel SM1^{T}.

The examination object is in the present case a human and the examination region includes the human's liver. Thus, in the present example the examination region corresponds to the reference region during the training of the machine-learning model as described with reference to FIG. 3.

The first representation R1 and the second representation R2 are radiological images; they may for example be MRI images and/or CT images and/or X-ray images.

The first representation R1 represents the examination region of the examination object without contrast agent or after administration of a first amount of a contrast agent. The second representation R2 represents the examination region of the examination object after administration of a second amount of the contrast agent. The second amount is larger than the first amount (it being possible also for the first amount to be zero, as described).

The trained first submodel SM1^{T} is configured and has been trained to determine, based on the first reference representation R1 and the second reference representation R2 and on the basis of model parameters, at least one model parameter MP for the second submodel SM2. The at least one model parameter MP is fed to the second submodel SM2. The second submodel SM2 is configured to generate, based on the first representation R1 and/or second representation R2 and based on the at least one model parameter MP, a synthetic third representation R3*.

The synthetic third representation R3* represents the examination region of the examination object after administration of a third amount of the contrast agent. The third amount is different from the second amount, preferably the third amount is larger than the second amount. The third amount is set by the second submodel SM2 and the at least one model parameter MP. The third amount depends on the purpose for which the trained machine-learning model M^{T} has been trained.

The synthetic third representation R3* can be outputted (for example displayed on a monitor or printed on a printer) and/or stored in a data storage medium and/or transmitted to a separate computer system.

It is also possible to discard the trained first submodel SM1^{T} after the training and to feed the first representation R1 and the second representation R2 directly to the second submodel SM2 in order to generate the synthetic third representation R3*. This applies in particular when it is often the same contrast enhancement that is to be achieved and the first amount, second amount and third amount of the contrast agent are to be the same for all examination objects and the at least one model parameter directly or indirectly indicates the third amount of the contrast agent. In this situation, the at least one model parameter MP determined after the training of the machine-learning model (M^{T}) can be inputted into the second submodel SM2 as a fixed parameter and does not need to be determined afresh for each set of new input data.

As already described, in addition to the first submodel and second submodel, the machine-learning model of the present disclosure may also comprise one or more further submodels.

In order to (further) reduce or eliminate noise and/or other unwanted artefacts in the synthetic third representation, the second submodel may be followed by a third submodel. The third submodel can serve for the correction of the synthetic third representation generated by the second submodel. The term “correction” can in this instance mean reducing or eliminating noise and/or artefacts.

The synthetic third representation generated by the second submodel can be fed to the third submodel as input data and, based on this input data and based on model parameters, the third submodel generates a corrected third representation. In addition to the synthetic third representations generated by the second submodel, further data can be fed to the third submodel as input data (see below).

The third submodel may be a machine-learning model. The third submodel may have been trained on the basis of training data to generate, based on a synthetic third representation generated by the second submodel and model parameters, a corrected (for example modified and/or optimized) third representation.

This is depicted in schematic form in FIG. 5.

FIG. 5 shows by way of example and in schematic form the training of a machine-learning model of the present disclosure.

The machine-learning model M comprises a first submodel SM1, a second submodel SM2 and a third submodel SM3.

All statements made above in relation to the training process shown in FIG. 3 similarly apply to the training process shown in FIG. 5, with the following differences:
The synthetic reference representation RR3* generated by the second submodel SM2 is fed to the third submodel SM3. In addition, the third submodel SM3 may also be fed with the first reference representation RR1 and/or the second reference representation RR2 and/or further input data.
The third submodel SM3 is configured and trained to generate, based on the input data it has been fed with and based on model parameters, a corrected third reference representation RR3*^{C}.
A loss function LF is used to quantify differences between the corrected third reference representation RR3*^{C} and the (measured) third reference representation RR3 of the training data. The differences can be minimized by modifying model parameters of the second submodel and/or of the third submodel in an optimization process (for example a gradient process).

The third submodel may for example be an artificial neural network or include such a network. The third submodel may be a convolutional neural network or include such a network.

The third submodel may have an autoencoder architecture, for example the third submodel may have an architecture such as the U-net (see for example O. Ronneberger et al.: U-net: Convolutional networks for biomedical image segmentation, International Conference on Medical image computing and computer-assisted intervention, pages 234-241, Springer, 2015, https://doi.org/10.1007/978-3-319-24574-4_28).

The third submodel may be a generative adversarial network (GAN) (see for example M.-Y. Liu et al.: Generative Adversarial Networks for Image and Video Synthesis: Algorithms and Applications, arXiv:2008.02793; J. Henry et al.: Pix2Pix GAN for Image-to-Image Translation, DOI: 10.13140/RG.2.2.32286.66887).

The third submodel may in particular be a generative adversarial network (GAN) for image super-resolution (SR) (see for example C. Ledig et al.: Photo-Realistic Single Image Super-Resolution Using a Generative Adversarial Network, arXiv:1609.04802v5).

The third submodel may be a transformer network (see for example D. Karimi et al.: Convolution-Free Medical Image Segmentation using Transformers, arXiv:2102.13645 [eess.IV]).

In the training process shown in FIG. 5, the first submodel SM1 and the third submodel SM3 are trained in tandem/concomitantly. The second submodel SM2 does not undergo training, because the model parameters MP of the second submodel SM2 are not learnable model parameters but are determined by the first submodel SM1. It is however also possible for the first submodel SM1 and the third submodel SM3 to be trained independently of one another. It is for example possible to first train the first submodel SM1 as described in relation to FIG. 3 and to then freeze the model parameters of the first submodel SM1. In a subsequent step, the third submodel SM3 then undergoes training. After training of the third submodel SM3, the overall model M can then undergo training in an end-to-end training process.

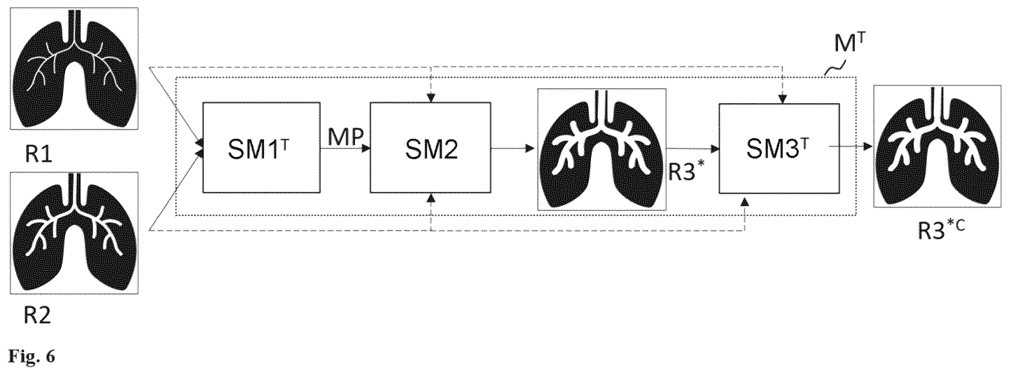

Once the machine-learning model M shown in FIG. 5 has been trained, it can then be used to generate a corrected synthetic representation of an examination region of an examination object. This is depicted by way of example and in schematic form in FIG. 6.

FIG. 6 shows by way of example and in schematic form the generation of a synthetic representation of an examination region of an examination object with the aid of a trained machine-learning model.

The trained machine-learning model M^{T} may for example have been trained as described in relation to FIG. 5. The superscripted T in the reference sign M^{T} for the machine-learning model M^{T} serves to indicate that this is a trained model. The same applies also to the first submodel SM1^{T} and the third submodel SM3^{T}.

The statements made above in relation to the process shown in FIG. 4 similarly apply also to the process shown in FIG. 6, with the following differences:
The synthetic third representation R3* generated by the second submodel SM2 is fed to the trained third submodel SM3^{T}. In addition, the trained third submodel SM3^{T} may also be fed with the first representation R1 and/or the second representation R2 of the examination region of the examination object and/or further input data.
The trained third submodel SM3^{T} is configured and has been trained to generate, based on the input data it has been fed with and model parameters, a corrected third representation R3*^{C}.
The corrected third representation R3*^{C} represents the examination region of the examination object after administration of the third amount of the contrast agent with less noise and/or fewer artefacts that the synthetic third representation R3*.
The corrected third representation R3*^{C} can be outputted (for example displayed on a monitor or printed on a printer) and/or stored in a data storage medium and/or transmitted to a separate computer system.

It is also possible with a machine-learning model of the present disclosure comprising a first submodel and a second submodel and optionally a third submodel to prepend an initial submodel to the first submodel.

It is for example possible for the initial submodel to perform a co-regression of the first (reference) representation and the second (reference) representation. It is for example possible for the initial submodel to perform a normalization and/or a segmentation and/or masking and/or another/a further transform operation/modification of the first (reference) representation and the second (reference) representation. For example, the initial submodel may carry out a Fourier transform or an inverse Fourier transform of the first and/or second (reference) representation.

Such an initial submodel may for example be an artificial neural network or include such a network.

If all submodels of the machine-learning model are differentiable, then it is possible for the machine-learning model to undergo training in an end-to-end training process.

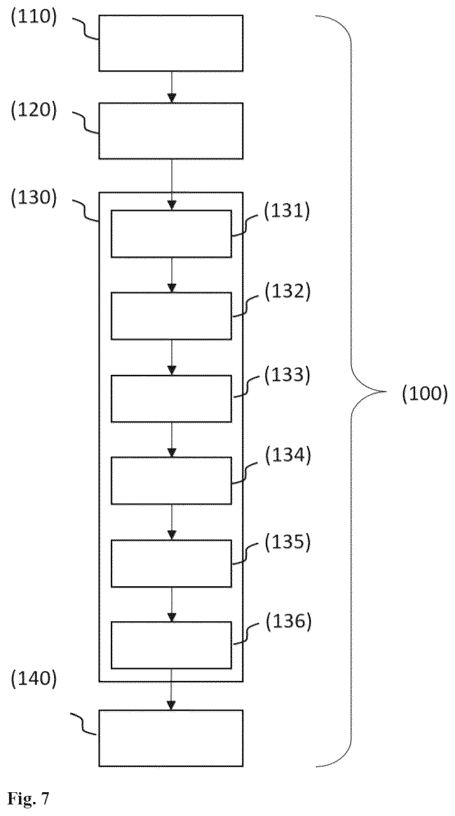

FIG. 7 shows an embodiment for the training of the machine-learning model in the form of a flowchart. The training process (100) comprises the steps of:
(110) providing training data, where the training data for each reference object of a multiplicity of reference objects comprise as target data: (i) a first reference representation of a reference region of the reference object and a second reference representation of the reference region of the reference object as input data and (ii) a third reference representation of the reference region of the reference object,
   where the first reference representation represents the reference region without contrast agent or after administration of a first amount of a contrast agent,
   where the second reference representation represents the reference region after administration of a second amount of the contrast agent, the second amount being larger than the first amount,
   where the third reference representation represents the reference region after administration of a third amount of the contrast agent, the third amount being different from the second amount, preferably larger than the second amount,
(120) providing a machine learning model, where the machine-learning model comprises two submodels: a first submodel and a second submodel.
(130) training the machine-learning model, where the training comprises the steps of:
(131) feeding a first reference representation and a second reference representation of a reference region of a reference object to the first submodel, where the first submodel is configured to determine, based on the first reference representation, on the second reference representation and on model parameters, at least one model parameter for the second submodel,
(132) receiving of the at least one model parameter determined by the first submodel,
(133) feeding the at least one model parameter that has been determined and the first reference representation and/or the second reference representation to the second submodel, where the second submodel is configured to generate, based on the first reference representation and/or the second reference representation and on the model parameter determined by the first submodel, a synthetic third reference representation,
(134) receiving of the synthetic third reference representation generated by the second submodel,
(135) quantifying the differences between the synthetic third reference representation and the third reference representation of the training data,
(136) reducing the differences by modifying model parameters of the first submodel,
(140) storing the trained machine-learning model and/or using the trained machine-learning model to generate a synthetic representation of an examination region of an examination object.

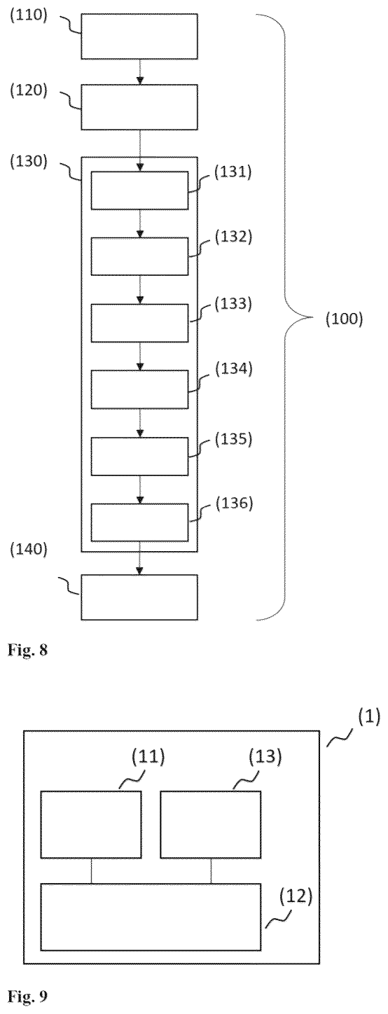

FIG. 8 shows an embodiment for the generation of a synthetic representation of an examination region of an examination object (prediction process) in the form of a flowchart.

The prediction process (200) comprises the steps of:
(210) providing a trained machine-learning model,
(220) providing a first representation, where the first representation represents an examination region of an examination object without contrast agent or after administration of a first amount of a contrast agent,
(230) providing a second representation, where the second representation represents the examination region of the examination object after administration of a second amount of the contrast agent, the second amount being larger than the first amount,
(240) feeding the first representation and the second representation to the trained machine-learning model, where the trained machine-learning model comprises two submodels: a first submodel and a second submodel, where the first submodel is configured and has been trained to determine, based on the first reference representation and second reference representation of a reference object, at least one model parameter for the second submodel and where the second submodel is configured to generate, based on the first reference representation and/or second reference representation and on the at least one model parameter determined by the first submodel, a synthetic third representation,
(250) receiving from the trained machine-learning model a synthetic third representation of the examination region of the examination object, where the synthetic third representation represents the reference region after administration of a third amount of the contrast agent, the third amount being different from the second amount, preferably larger than the second amount,
(260) outputting and/or storing the synthetic third representation and/or transmitting the synthetic third representation to a separate computer system.

The term “providing” can for example denote “receiving” or “generating”.

The term “receiving” encompasses both the retrieving and the accepting of subject matters (for example of representations and/or of a (trained) machine-learning model) that are transmitted for example to the computer system of the present disclosure. Subject matters may be read from one or more data storage media and/or transmitted from a separate computer system. Representations may be received for example from a computed tomography system, from a magnetic resonance imaging system or from an ultrasound scanner.

The term “generating” as used herein may mean that a representation is generated on the basis of another (for example a received) representation or on the basis of a plurality of other (for example received) representations. For example, a received representation may be a representation of an examination region of an examination object in real space. On the basis of this real-space representation it is possible for example to generate a representation of the examination region of the examination object in frequency space through a transform operation (for example a Fourier transform). Further options for generating a representation based on one or more other representations are described in this description.

FIG. 9 shows by way of example and in schematic form a computer system according to the present disclosure.

A “computer system” is an electronic data processing system that processes data by means of programmable calculation rules. Such a system typically comprises a “computer”, which is the unit that includes a processor for carrying out logic operations, and peripherals.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (1) shown in FIG. 9 comprises a receiving unit (11), a control and calculation unit (12) and an output unit (13).

The control and calculation unit (12) serves for control of the computer system (1), coordination of the data flows between the units of the computer system (1), and for the performance of calculations.

The control and calculation unit (12) is configured:
to generate a first representation or cause the receiving unit (11) to receive a first representation, where the first representation represents an examination region of an examination object without contrast agent or after administration of a first amount of a contrast agent,
to generate a second representation or to cause the receiving unit (11) to receive a second representation, where the second representation represents the examination region of the examination object after administration of a second amount of the contrast agent, the second amount being larger than the first amount,
to feed the first representation and the second representation to a trained machine-learning model,
   where the trained machine-learning model has been trained on the basis of training data,
   where the training data for each reference object of a multiplicity of reference objects comprise as target data: (i) a first reference representation of a reference region of the reference object and a second reference representation of the reference region of the reference object as input data and (ii) a third reference representation of the reference region of the reference object,
   where the first reference representation represents the reference region without contrast agent or after administration of a first amount of a contrast agent,
   where the second reference representation represents the reference region after administration of a second amount of the contrast agent, the second amount being larger than the first amount,
   where the third reference representation represents the reference region after administration of a third amount of the contrast agent, the third amount being different from the second amount, preferably larger than the second amount,
   where the trained machine-learning model comprises two submodels: a first submodel and a second submodel, where the first submodel is configured and has been trained to determine, based on the first reference representation and second reference representation of a reference object, at least one model parameter for the second submodel and where the second submodel is configured to generate, based on the first reference representation and/or second reference representation and on the at least one model parameter determined by the first submodel, a synthetic third representation,
to receive from the trained machine-learning model a synthetic third representation of the examination region of the examination object, where the synthetic third representation represents the reference region after administration of a third amount of the contrast agent, the third amount being different from the second amount, preferably larger than the second amount,
to cause the output unit (13) to output the synthetic third representation and/or to store it in a data storage medium and/or to transmit it to a separate computer system.

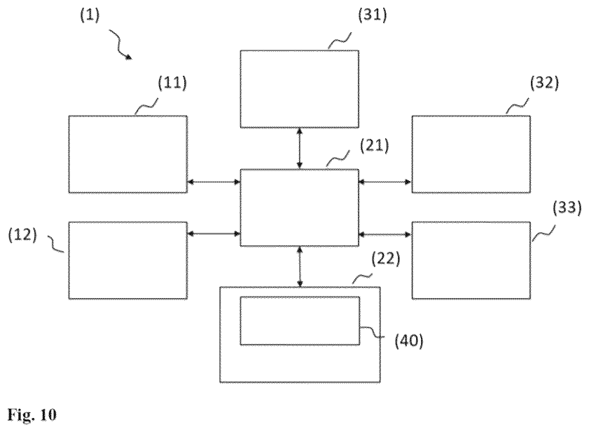

FIG. 10 shows by way of example and in schematic form a further embodiment of the computer system. The computer system (1) comprises a processing unit (21) connected to a storage medium (22). The processing unit (21) and the storage medium (22) form a control and calculation unit, as shown in FIG. 9.

The processing unit (21) may comprise one or more processors alone or in combination with one or more storage media. The processing unit (21) may be customary computer hardware that is able to process information such as digital images, computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the storage medium (22) of the same or of a different computer system.

The storage medium (22) may be customary computer hardware that is able to store information such as digital images (for example representations of the examination region), data, computer programs and/or other digital information either temporarily and/or permanently. The storage medium (22) may comprise a volatile and/or non-volatile storage medium and may be fixed in place or removable. Examples of suitable storage media are RAM (random access memory), ROM (read-only memory), a hard disk, a flash memory, an exchangeable computer floppy disk, an optical disc, a magnetic tape or a combination of the aforementioned. Optical discs can include compact discs with read-only memory (CD-ROM), compact discs with read/write function (CD-R/W), DVDs, Blu-ray discs and the like.

The processing unit (21) may be connected not just to the storage medium (22), but also to one or more interfaces (11, 12, 31, 32, 33) in order to display, transmit and/or receive information. The interfaces may comprise one or more communication interfaces (11, 32, 33) and/or one or more user interfaces (12, 31). The one or more communication interfaces may be configured to send and/or receive information, for example to and/or from an MRI scanner, a CT scanner, an ultrasound camera, other computer systems, networks, data storage media or the like. The one or more communication interfaces may be configured to transmit and/or receive information via physical (wired) and/or wireless communication connections. The one or more communication interfaces may comprise one or more interfaces for connection to a network, for example using technologies such as mobile telephone, wifi, satellite, cable, DSL, optical fibre and/or the like. In some examples, the one or more communication interfaces may comprise one or more close-range communication interfaces configured to connect devices having close-range communication technologies such as NFC, RFID, Bluetooth, Bluetooth LE, ZigBee, infrared (e.g. IrDA) or the like.

The user interfaces may include a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image- or video-recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces may in addition comprise one or more interfaces for communication with peripherals such as printers and the like.

One or more computer programs (40) may be stored in the storage medium (22) and executed by the processing unit (21), which is thereby programmed to fulfil the functions described in this description. The retrieving, loading and execution of instructions of the computer program (40) may take place sequentially, such that an instruction is respectively retrieved, loaded and executed. However, the retrieving, loading and/or execution may also take place in parallel.

The computer system of the present disclosure may be designed as a laptop, notebook, netbook and/or tablet PC; it may also be a component of an MRI scanner, a CT scanner or an ultrasound diagnostic device.

The present disclosure also provides a computer program product. Such a computer program product includes a non-volatile data carrier, for example a CD, a DVD, a USB stick or another data storage medium. Stored on the data carrier is a computer program. The computer program can be loaded into a working memory of a computer system (more particularly into a working memory of a computer system of the present disclosure), where it causes the computer system to execute the following steps:
receiving or generating a first representation, where the first representation represents an examination region of an examination object without contrast agent or after administration of a first amount of a contrast agent,
receiving or generating a second representation, where the second representation represents the examination region of the examination object after administration of a second amount of the contrast agent, the second amount being larger than the first amount,
feeding the first representation and the second representation to a trained machine-learning model, where the trained machine-learning model has been trained on the basis of training data,
   where the training data for each reference object of a multiplicity of reference objects comprise as target data: (i) a first reference representation of a reference region of the reference object and a second reference representation of the reference region of the reference object as input data and (ii) a third reference representation of the reference region of the reference object,
   where the first reference representation represents the reference region without contrast agent or after administration of a first amount of a contrast agent,
   where the second reference representation represents the reference region after administration of a second amount of the contrast agent, the second amount being larger than the first amount,
   where the third reference representation represents the reference region after administration of a third amount of the contrast agent, the third amount being different from the second amount, preferably larger than the second amount,
   where the trained machine-learning model comprises two submodels: a first submodel and a second submodel, where the first submodel is configured and has been trained to determine, based on the first reference representation and second reference representation of a reference object, at least one model parameter for the second submodel and where the second submodel is configured to generate, based on the first reference representation and/or second reference representation and on the at least one model parameter determined by the first submodel, a synthetic third representation,
receiving from the trained machine-learning model a synthetic third representation of the examination region of the examination object, where the synthetic third representation represents the reference region after administration of a third amount of the contrast agent, the third amount being different from the second amount, preferably larger than the second amount,
outputting and/or storing the synthetic third representation and/or transmitting the synthetic third representation to a separate computer system.

The computer program product can also be marketed in combination (in a set) with the contrast agent. Such a set is also referred to as a kit. Such a kit comprises the contrast agent and the computer program product. It is also possible for such a kit to comprise the contrast agent and means that allow the purchaser to obtain the computer program, for example by downloading it from a webpage. These means may include a link, i.e. an address of the webpage on which the computer program can be obtained, for example from which the computer program can be downloaded to a computer system connected to the internet. These means may include a code (for example an alphanumeric string or a QR code, or a DataMatrix code or a barcode or another optically and/or electronically readable code) that gives the purchaser access to the computer program. Such a link and/or code many for example be printed on a packaging of the contrast agent and/or on a package leaflet of the contrast agent. A kit is thus a combination product comprising a contrast agent and a computer program (for example in the form of access to the computer program or in the form of executable program code on a data carrier) that are offered for sale together.

Embodiments of the present disclosure are:

1. A computer-implemented method comprising:
providing a trained machine-learning model,
   wherein the trained machine-learning model has been trained on the basis of training data,
   wherein the training data for each reference object of a plurality of reference objects comprise as target data: (i) a first reference representation of a reference region of the reference object and a second reference representation of the reference region of the reference object as input data and (ii) a third reference representation of the reference region of the reference object,
   wherein the first reference representation represents the reference region without contrast agent or after administration of a first amount of a contrast agent,
   wherein the second reference representation represents the reference region after administration of a second amount of the contrast agent, the second amount being larger than the first amount,
   wherein the third reference representation represents the reference region after administration of a third amount of the contrast agent, the third amount being different from the second amount, preferably larger than the second amount,
   wherein the trained machine-learning model comprises two submodels: a first submodel and a second submodel, where the first submodel is configured and has been trained to determine, based on the first reference representation and second reference representation of a reference object, at least one model parameter for the second submodel and where the second submodel is configured to generate, based on the first reference representation and/or second reference representation and on the at least one model parameter determined by the first submodel, a synthetic third representation,
providing a first representation, wherein the first representation represents an examination region of an examination object without contrast agent or after administration of a first amount of a contrast agent,
providing a second representation, wherein the second representation represents the examination region of the examination object after administration of a second amount of the contrast agent, the second amount being larger than the first amount,
feeding the first representation and the second representation to the trained machine-learning model,
receiving from the trained machine-learning model a synthetic third representation of the examination region of the examination object, wherein the synthetic third representation represents the reference region after administration of a third amount of the contrast agent, the third amount being different from the second amount, preferably larger than the second amount,
outputting and/or storing the synthetic third representation and/or transmitting the synthetic third representation to a separate computer system.

2. The method according to embodiment 1, wherein the second submodel comprises:
subtracting the first representation of the examination region of the examination object from the second representation of the examination region of the examination object, wherein a difference is formed,
multiplying the difference by a gain factor, wherein the gain factor is a positive or negative real number and wherein the at least one model parameter determined by the first submodel includes the gain factor,
adding the difference multiplied by the gain factor to the first representation or the second representation.

3. The method according to either of embodiments 1 or 2, wherein the first representation and the second representation are representations of the examination region of the examination object in frequency space, wherein the second submodel comprises:
subtracting the first representation of the examination region of the examination object from the second representation of the examination region of the examination object, wherein a difference is formed,
multiplying the difference by a frequency-dependent weight function, wherein a weighted difference is formed, wherein the at least one model parameter determined by the first submodel includes one or more parameters of the frequency-dependent weight function,
multiplying the weighted difference by a gain factor, wherein the gain factor is a positive or negative real number and wherein the at least one model parameter determined by the first submodel preferably includes the gain factor,
adding the weighted difference multiplied by the gain factor to the first representation or the second representation.

4. The method according to any of embodiments 1 to 3, wherein the first submodel is an artificial neural network or includes such a network.

5. The method according to any of embodiments 1 to 4, wherein the second submodel is a mechanistic model.

6. The method according to any of embodiments 1 to 5, wherein model parameters of the second submodel are not trainable parameters.

7. The method according to any of embodiments 1 to 6, wherein the machine-learning model is differentiable and the training takes place in an end-to-end manner.

8. The method according to any of embodiments 1 to 7, wherein the machine-learning model includes a third submodel, wherein the third submodel is configured and has been trained to generate, based on the synthetic third representation generated by the second submodel, a corrected third representation, wherein the step of receiving from the trained machine-learning model a synthetic third representation of the examination region of the examination object comprises: receiving from a trained machine-learning model a corrected third representation of the examination region of the examination object, wherein the corrected third representation represents the reference region after administration of the third amount of the contrast agent and wherein the step of outputting and/or storing the synthetic third representation and/or transmitting the synthetic third representation to a separate computer system comprises: outputting and/or storing the corrected third representation and/or transmitting the corrected third representation to a separate computer system.

9. The method according to any of embodiments 1 to 8, wherein the third submodel is a trained machine-learning model.

10. The method according to any of embodiments 1 to 9, wherein the third submodel is an artificial neural network or includes such a network.

11. The method according to any of embodiments 1 to 10, wherein the machine-learning model includes an initial submodel prepended to the first submodel, where the initial submodel is configured to carry out a co-registration of the first representation and the second representation and/or to carry out a segmentation on the first representation and/or on the second representation and/or to carry out a normalization on the first representation and/or on the second representation and/or to carry out a transform operation on the first and/or on the second representation.

12. The method according to any of embodiments 1 to 11, wherein the training of the machine-learning model comprises:
providing the training data,
providing a machine-learning model,
training the machine-learning model, wherein the training comprises:
   feeding a first reference representation and a second reference representation of a reference region of a reference object to the first submodel, wherein the first submodel is configured to determine, based on the first reference representation, on the second reference representation and on model parameters, at least one model parameter for the second submodel,
   receiving of the at least one model parameter determined by the first submodel,
   feeding the at least one model parameter that has been determined and the first reference representation and/or the second reference representation to the second submodel, wherein the second submodel is configured to generate, based on the first reference representation and/or the second reference representation and on the model parameter determined by the first submodel, a synthetic third reference representation,
   receiving of the synthetic third reference representation generated by the second submodel,
   quantifying the differences between the synthetic third reference representation and the third reference representation of the training data,
   reducing the differences by modifying model parameters of the first submodel,
storing the trained machine-learning model and/or using the trained machine-learning model to generate a synthetic representation of an examination region of an examination object.

13. The method according to any of embodiments 1 to 12, wherein the training of the machine-learning model comprises:
providing the training data,
providing a machine-learning model, wherein the machine-learning model includes a third submodel,
training the machine-learning model, where the training comprises:
   feeding a first reference representation and a second reference representation of a reference region of a reference object to the first submodel, wherein the first submodel is configured to determine, based on the first reference representation, on the second reference representation and on model parameters, at least one model parameter for the second submodel,
   receiving of the at least one model parameter determined by the first submodel,
   feeding the at least one model parameter that has been determined and the first reference representation and/or the second reference representation to the second submodel, wherein the second submodel is configured to generate, based on the first reference representation and/or the second reference representation and on the model parameter determined by the first submodel, a synthetic third reference representation,
   receiving of the synthetic third reference representation generated by the second submodel,
   feeding the synthetic third reference representation generated by the second submodel to the third submodel, wherein the third submodel is configured to generate, based on the synthetic third reference representation generated by the second submodel and on model parameters, a corrected third reference representation,
   receiving of the corrected third reference representation generated by the third submodel,
   quantifying the differences between the corrected third reference representation and the third reference representation of the training data,
   reducing the differences by modifying model parameters of the first and/or third submodel,
storing the trained machine-learning model and/or using the trained machine-learning model to generate a synthetic representation of an examination region of an examination object.

14. The method according to any of embodiments 1 to 13, further comprising: during and/or after the training of the machine-learning model:
outputting of the at least one model parameter.

15. The method according to any of embodiments 1 to 14, further comprising: during and/or after the training of the machine-learning model:
checking whether the at least one model parameter is above an upper predefined limit value or below a lower predefined limit value,
in the event that the at least one model parameter is above an upper predefined limit value or below a lower predefined limit value: issuing a message stating that the model stays within the specified limits or goes outside them.

16. The method according to any of embodiments 1 to 15, wherein the frequency-dependent weight function is a Hann function, a Poisson function, or a Gaussian function

17. The method according to any of embodiments 1 to 16, wherein the examination object is a human and the examination region is a part of the human.

18. The method according to any of embodiments 1 to 17, wherein each reference object is a human and the reference region of each such reference object is a part of the reference object.

19. The method according to any of embodiments 1 to 18, wherein the reference region of each such reference object and the examination region are the same part of the human.

20. The method according to any of embodiments 1 to 19, wherein each representation and each reference representation is the result of a radiological examination.

21. The method according to any of embodiments 1 to 20, wherein the radiological examination is an MRI examination or a CT examination.

22. The method according to any of embodiments 1 to 21, wherein the contrast agent is an MRI contrast agent.

23. The method according to any of embodiments 1 to 22, wherein the examination region and the reference region of each such reference object include the liver.

24. The method according to any of embodiments 1 to 23, wherein the contrast agent is a hepatobiliary contrast agent.

25. The method according to any of embodiments 1 to 24, wherein the first amount is equal to zero, the second amount is smaller than a standard amount of the contrast agent and the third amount is equal to the standard amount of the contrast agent.

26. The method according to any of embodiments 1 to 25, wherein the first amount is equal to zero, the second amount is smaller than the standard amount of the contrast agent or equal to the standard amount of the contrast agent and the third amount is larger than the standard amount of the contrast agent.

27. Device/system for data processing, comprising a processor that is adapted/configured for executing the method of any of embodiments 1 to 26.

28. Computer system comprising means for executing the method of any of embodiments 1 to 26.

29. Computer program product comprising commands that, when the program is executed by a computer, cause the computer to execute the method of any of embodiments 1 to 26.

30. Computer-readable storage medium comprising commands that, when executed by a computer, cause the computer to execute the method of any of embodiments 1 to 26.

32. Kit comprising a computer program or an access to a computer program and a contrast agent, wherein the computer program can be loaded into the working memory of a computer system, where it causes the computer system to execute the method of any of embodiments 1 to 26.

33. Use of a contrast agent in a radiological examination method, where the radiological examination method comprises the method of any of embodiments 1 to 26.

34. Contrast agent for use in a radiological examination method, where the radiological examination method comprises the method of any of embodiments 1 to 26.

The present disclosure can be used for various purposes. Some examples of use are described below, without the disclosure being limited to these examples of use.

A first example of use concerns magnetic resonance imaging examinations for differentiating intraaxial tumours such as intracerebral metastases and malignant gliomas. The infiltrative growth of these tumours makes it difficult to differentiate exactly between tumour and healthy tissue. Determining the extent of a tumour is however crucial for surgical removal. Distinguishing between tumours and healthy tissue is facilitated by administration of an extracellular MRI contrast agent; after intravenous administration of a standard dose of 0.1 mmol/kg body weight of the extracellular MRI contrast agent gadobutrol, intraaxial tumours can be differentiated much more readily. At higher doses, the contrast between lesion and healthy brain tissue is increased further; the detection rate of brain metastases increases linearly with the dose of the contrast agent (see for example M. Hartmann et al.: Does the administration of a high dose of a paramagnetic contrast medium (Gadovist) improve the diagnostic value of magnetic resonance tomography in glioblastomas? doi: 10.1055/s-2007-1015623).

A single triple dose or a second subsequent dose may be administered here up to a total dose of 0.3 mmol/kg body weight. This exposes the patient and the environment to additional gadolinium and in the case of a second scan, incurs additional costs.

The present disclosure can be used to avoid the dose of contrast agent exceeding the standard amount. A first MRI image can be generated without contrast agent or with less than the standard amount and a second MRI image generated with the standard amount. On the basis of these generated MRI images it is possible, as described in this disclosure, to generate a synthetic MRI image in which the contrast between lesions and healthy tissue can be varied within wide limits by altering the gain factor α. This makes it possible to achieve contrasts that are otherwise achieved by administering an amount of contrast agent larger than the standard amount.

Another example of use concerns the reduction of the amount of MRI contrast agent in a magnetic resonance imaging examination. Gadolinium-containing contrast agents such as gadobutrol are used for a diversity of examinations. They are used for contrast enhancement in examinations of the cranium, spine, chest or other examinations. In the central nervous system, gadobutrol highlights regions where the blood-brain barrier is impaired and/or abnormal vessels. In breast tissue, gadobutrol makes it possible to visualize the presence and extent of malignant breast disease. Gadobutrol is also used in contrast-enhanced magnetic resonance angiography for diagnosing stroke, for detecting tumour blood perfusion and for detecting focal cerebral ischaemia.

Increasing environmental pollution, the cost burden on the health system and the fear of acute side effects and possible long-term health risks, especially in the case of repeated and long-term exposure, have given impetus to efforts to reduce the dose of gadolinium-containing contrast agents. This can be achieved by the present disclosure.

A first MRI image without contrast agent and a second MRI image with an amount of contrast agent less than the standard amount can be generated. On the basis of these generated MRI images it is possible, as described in this disclosure, to generate a synthetic MRI image in which the contrast can be varied within wide limits by altering the gain factor α. This makes it possible with less than the standard amount of contrast agent to achieve the same contrast as is obtained after administration of the standard amount.

Another example of use concerns the detection, identification and/or characterization of lesions in the liver with the aid of a hepatobiliary contrast agent such as Primovist®.

Primovist® is administered intravenously (i.v.) at a standard dose of 0.025 mmol/kg body weight. This standard dose is lower than the standard dose of 0.1 mmol/kg body weight in the case of extracellular MRI contrast agents. Unlike in contrast-enhanced MRI with extracellular gadolinium-containing contrast agents, Primovist® permits dynamic multiphase T1w imaging. However, the lower dose of Primovist® and the observation of transient motion artefacts that can occur shortly after intravenous administration, means that contrast enhancement with Primovist® in the arterial phase is perceived by radiologists as poorer than contrast enhancement with extracellular MRI contrast agents. The assessment of contrast enhancement in the arterial phase and of the vascularity of focal liver lesions is however of critical importance for accurate characterization of the lesion.

With the aid of the present disclosure it is possible to increase contrast, particularly in the arterial phase, without the need to administer a higher dose.

A first MRI image without contrast agent and a second MRI image during the arterial phase after administering an amount of a contrast agent that corresponds to the standard amount can be generated. On the basis of these generated MRI images it is possible, as described in this disclosure, to generate a synthetic MRI image in which the contrast in the arterial phase can be varied within wide limits by altering the gain factor α. This makes it possible to achieve contrasts that are otherwise achieved by administering an amount of contrast agent larger than the standard amount.

Another example of use concerns the use of MRI contrast agents in computed tomography examinations.

In a CT examination, MRI contrast agents usually have a lower contrast-enhancing effect than CT contrast agents. However, it can be advantageous to employ an MRI contrast agent in a CT examination. An example is a minimally invasive intervention in the liver of a patient in whom a surgeon is monitoring the procedure by means of a CT scanner. Computed tomography (CT) has the advantage over magnetic resonance imaging that more major surgical interventions are possible in the examination region while generating CT images of an examination region of an examination object. On the other hand, there are few surgical instruments and surgical devices that are MRI-compatible. Moreover, access to the patient is restricted by the magnets used in MRI. Thus, while a surgeon is performing an operation in the examination region, he/she is able to use CT to visualize the examination region and to monitor the operation.

For example, if a surgeon wishes to perform a procedure in a patient's liver in order for example to carry out a biopsy on a liver lesion or to remove a tumour, the contrast between a liver lesion or tumour and healthy liver tissue will not be as pronounced in a CT image of the liver as it is in an MRI image after administration of a hepatobiliary contrast agent. No CT-specific hepatobiliary contrast agents are currently known and/or approved in CT. The use of an MRI contrast agent, more particularly a hepatobiliary MRI contrast agent, in computed tomography thus combines the possibility of differentiating between healthy and diseased liver tissue and the possibility of carrying out an operation with simultaneous visualization of the liver.

The comparatively low contrast enhancement achieved by the MRI contrast agent can be increased with the aid of the present disclosure without the need to administer a dose higher than the standard dose.

A first CT image without MRI contrast agent and a second CT image after administering an amount of a MRI contrast agent that corresponds to the standard amount can be generated. On the basis of these generated CT images it is possible, as described in this disclosure, to generate a synthetic CT image in which the contrast produced by the MRI contrast agent can be varied within wide limits by altering the gain factor α. This makes it possible to achieve contrasts that are otherwise achieved by administering an amount of MRI contrast agent larger than the standard amount.

1. A computer-implemented method comprising:
providing a trained machine-learning model (M^{T});
wherein the trained machine-learning model (M^{T}) has been trained on the basis of training data (TD),
wherein the training data (TD) for each reference object of a plurality of reference objects comprise as target data: (i) a first reference representation (RR1) of a reference region of the reference object and a second reference representation (RR2) of the reference region of the reference object as input data and (ii) a third reference representation (RR3) of the reference region of the reference object,
wherein the first reference representation (RR1) represents the reference region without contrast agent or after administration of a first amount of a contrast agent,
wherein the second reference representation (RR2) represents the reference region after administration of a second amount of the contrast agent, the second amount being larger than the first amount,
wherein the third reference representation (RR3) represents the reference region after administration of a third amount of the contrast agent, the third amount being different from the second amount,
wherein the trained machine-learning model (M^{T}) comprises two submodels: a first submodel (SM1^{T}) and a second submodel (SM2), wherein the first submodel (SM1^{T}) is configured and has been trained to determine, based on the first reference representation (RR1) and second reference representation (RR2) of an examination region of a reference object, at least one model parameter for the second submodel (SM2) and wherein the second submodel (SM2) is configured to generate, based on at least one of the first reference representation (RR1) and second reference representation (RR2) and on the at least one model parameter (MP) determined by the first submodel (SM1^{T}), a synthetic third representation (RR3*);
providing a first representation (R1), wherein the first representation (R1) represents an examination region of an examination object without contrast agent or after administration of a first amount of a contrast agent;
providing a second representation (R2), wherein the second representation (R2) represents the examination region of the examination object after administration of a second amount of the contrast agent, the second amount being larger than the first amount;
feeding the first representation (R1) and the second representation (R2) to the trained machine-learning model (M^{T});
receiving from the trained machine-learning model (M^{T}) a synthetic third representation (R3*) of the examination region of the examination object, wherein the synthetic third representation (R3*) represents the reference region after administration of a third amount of the contrast agent, the third amount being different from the second amount; and
outputting and storing the synthetic third representation (R3*) and transmitting the synthetic third representation (R3*) to a separate computer system.

2. The method according to claim 1, wherein the second submodel (SM2) comprises:
subtracting the first representation (R1) of the examination region of the examination object from the second representation (R2) of the examination region of the examination object, wherein a difference (R2−R1) is formed;
multiplying the difference (R2−R1) by a gain factor (α), wherein the gain factor (α) is a positive or negative real number and wherein the at least one model parameter (MP) determined by the first submodel (SM1^{T}) includes the gain factor (α); and
adding the difference (R2−R1) multiplied by the gain factor (α) to the first representation (R1) or the second representation (R2).

3. The method according to claim 1, wherein the first representation (R1) and the second representation (R2) are representations (R1^{F}, R2^{F}) of the examination region of the examination object in frequency space, wherein the second submodel (SM2) comprises:
subtracting the first representation (R1^{F}) of the examination region of the examination object from the second representation (R2^{F}) of the examination region of the examination object, wherein a difference (R2^{F}−R1^{F}) is formed;
multiplying the difference (R2^{F}−R1^{F}) by a frequency-dependent weight function (WF), wherein a weighted difference (R2^{F}− R1^{F})^{W} is formed, wherein the at least one model parameter (MP) determined by the first submodel (SM1^{T}) includes one or more parameters of the frequency-dependent weight function (WF);
multiplying the weighted difference (R2^{F}−R1^{F})^{W} by a gain factor (α), wherein the gain factor (α) is a positive or negative real number and wherein the at least one model parameter (MP) determined by the first submodel (SM1^{T}) includes the gain factor (α); and
adding the weighted difference (R2^{F}−R1^{F})^{W} multiplied by the gain factor (α) to the first representation (R1^{F}) or the second representation (R2^{F}).

4. The method according to claim 1, wherein the first submodel (SM1^{T}) is an artificial neural network or includes such a network.

5. The method according to claim 1, wherein the second submodel (SM2) is a mechanistic model.

6. The method according to claim 1, wherein model parameters of the second submodel (SM2) are not trainable parameters.

7. The method according to claim 1, wherein the machine-learning model (M^{T}) is differentiable and the training takes place in an end-to-end manner.

8. The method according to claim 1, wherein the machine-learning model (M^{T}) includes a third submodel (SM3^{T}), wherein the third submodel (SM3^{T}) is configured and has been trained to generate, based on the synthetic third representation (R3*) generated by the second submodel (SM2), a corrected third representation (R3*^{C}), wherein the step of receiving from the trained machine-learning model (M) a synthetic third representation (R3*) of the examination region of the examination object comprises: receiving from the trained machine-learning model (M^{T}) the corrected third representation (R3*^{C}) of the examination region of the examination object, wherein the corrected third representation (R3*^{C}) represents the reference region after administration of the third amount of the contrast agent and wherein the step of outputting and storing the synthetic third representation (R3*) and transmitting the synthetic third representation (R3*) to a separate computer system comprises: outputting and storing the corrected third representation (R3*^{C}) and transmitting the corrected third representation (R3*^{C}) to a separate computer system.

9. The method according to claim 8, wherein the third submodel (SM3^{T}) is a trained machine-learning model and wherein the third submodel (SM3^{T}) is an artificial neural network or includes such a network.

10. The method according to claim 1, wherein the trained machine-learning model (M^{T}) has been trained and wherein the training of the trained machine-learning model (M^{T}) comprises:
providing the training data (TD);
providing a machine-learning model (M);
training the machine-learning model (M), wherein the training comprises:
feeding a first reference representation (RR1) and a second reference representation (RR2) of a reference region of a reference object to the first submodel (SM1), wherein the first submodel (SM1) is configured to determine, based on the first reference representation (RR1), on the second reference representation (RR2) and on model parameters, at least one model parameter (MP) for the second submodel (SM2);
receiving of the at least one model parameter (MP) determined by the first submodel (SM1);
feeding the at least one model parameter (MP) that has been determined and the first reference representation (RR1) and the second reference representation (RR2) to the second submodel (SM2), wherein the second submodel (SM2) is configured to generate, based on at least one of the first reference representation (RR1) and the second reference representation (RR2) and on the model parameter (MP) determined by the first submodel (SM1), a synthetic third reference representation (RR3*);
receiving of the synthetic third reference representation (RR3*) generated by the second submodel (SM2);
quantifying the differences between the synthetic third reference representation (RR3*) and the third reference representation (RR3) of the training data;
reducing the differences by modifying model parameters of the first submodel (SM1); and
storing the trained machine-learning model (M^{T}) and using the trained machine-learning model (M^{T}) to generate a synthetic representation (R3*) of an examination region of an examination object.

11. The method according to claim 3, wherein the frequency-dependent weight function (WF) is a Hann function, a Poisson function, or a Gaussian function.

12. The method according to claim 1, wherein the examination object is a human and the examination region is a part of the human, wherein each reference object is a human and the reference region of each such reference object is a part of the reference object and wherein the reference region of each such reference object and the examination region are the same part of the human.

13. The method according to any of claim 1, wherein the first representation (R1) and the second representation (R2) and also each reference representation (RR1, RR2, RR3) of one such reference object is a result of a radiological examination, wherein the radiological examination is an MRI examination or a CT examination and the contrast agent is an MRI contrast agent or a CT contrast agent.

14. The method according to claim 1, wherein the first amount is equal to zero, the second amount is smaller than a standard amount of the contrast agent or equal to the standard amount of the contrast agent and the third amount is larger than the standard amount of the contrast agent.

15. The method according to claim 1, wherein the contrast agent comprises:
a Gd³⁺ complex of a compound of formula (I) 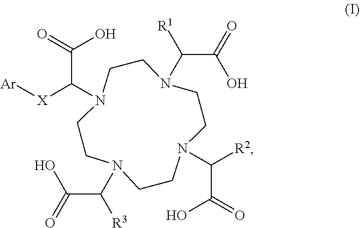
wherein:
Ar is a group selected from: 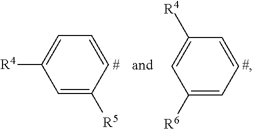
wherein _{#} is a linkage to X;
X is a group selected from:
CH₂, (CH₂)₂, (CH₂)₃, (CH₂)₄ and *—(CH₂)₂O—CH₂—^{#};
wherein * is a linkage to Ar and ^{#} is a linkage to an acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom;
and
R⁶ is a hydrogen atom;
or a stereoisomer, a tautomer, a hydrate, a solvate or a salt thereof, or a mixture thereof, or
a Gd³⁺ complex of a compound of formula (II) 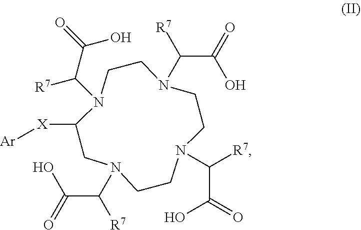
wherein:
Ar is a group selected from: 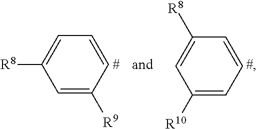
wherein _{#} is a linkage to X;
X is a group selected from CH₂, (CH₂)₂, (CH₂)₃, (CH₂)₄ and *—(CH₂)₂O—CH₂—^{#};
wherein * is a linkage to Ar and ^{#} is a linkage to an acetic acid residue;
R⁷ is a hydrogen atom or a group selected from C₁-C₃ alkyl, —CH₂OH, —(CH₂)₂OH and —CH₂OCH₃;
R⁸ is a group selected from:
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are independently a hydrogen atom;
or a stereoisomer, a tautomer, a hydrate, a solvate or a salt thereof, or a mixture thereof, or the contrast agent comprises one of the following substances:
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(±)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate;
gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3 S)-1,3,4-trihydroxybutan-2-yl)-1,4,7,10-tetraazacyclododecane-1,4,7-triyl)triacetate;
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium-2,2′,2″-[10-(carboxymethyl)-2-(4-ethoxybenzyl)-1,4,7,10-tetraazacyclododecane-1,4,7-triyl]triacetate.

16. A computer system comprising:
a receiving unit;
a control and calculation unit; and
an output unit;
wherein the control and calculation unit is configured to:
generate a first representation (R1) or cause the receiving unit to receive a first representation (R1), wherein the first representation (R1) represents an examination region of an examination object without contrast agent or after administration of a first amount of a contrast agent;
generate a second representation (R2) or to cause the receiving unit to receive a second representation (R2), wherein the second representation (R2) represents the examination region of the examination object after administration of a second amount of the contrast agent, the second amount being larger than the first amount;
feed the first representation (R1) and the second representation (R2) to a trained machine-learning model (M^{T});
wherein the trained machine-learning model (M^{T}) has been trained on the basis of training data (TD),
wherein the training data (TD) for each reference object of a plurality of reference objects comprise as target data: (i) a first reference representation (RR1) of a reference region of the reference object and a second reference representation (RR2) of the reference region of the reference object as input data and (ii) a third reference representation (RR3) of the reference region of the reference object,
wherein the first reference representation (RR1) represents the reference region without contrast agent or after administration of a first amount of a contrast agent,
wherein the second reference representation (RR2) represents the reference region after administration of a second amount of the contrast agent, the second amount being larger than the first amount,
wherein the third reference representation (RR3) represents the reference region after administration of a third amount of the contrast agent, the third amount being different from the second amount,
wherein the trained machine-learning model (M^{T}) comprises two submodels: a first submodel (SM1^{T}) and a second submodel (SM2), wherein the first submodel (SM1^{T}) is configured and has been trained to determine, based on the first reference representation (RR1) and second reference representation (RR2) of each such reference object, at least one model parameter (MP) for the second submodel (SM2) and wherein the second submodel (SM2) is configured to generate, based on at least one of the first reference representation (RR1) and second reference representation (RR2) and on the at least one model parameter (MP) determined by the first submodel (SM1^{T}), a synthetic third representation (RR3*);
receive from the trained machine-learning model (M^{T}) a synthetic third representation (R3*) of the examination region of the examination object, wherein the synthetic third representation (R3*) represents the reference region after administration of a third amount of the contrast agent, the third amount being different from the second amount; and
cause the output unit to output the synthetic third representation (R3*) and to store it in a data storage medium and to transmit it to a separate computer system.

17. A computer program product comprising a data carrier on which there is stored a computer program that can be loaded into a working memory of a computer system, wherein the computer program causes the computer system to:
provide a trained machine-learning model (M^{T});
wherein the trained machine-learning model (M^{T}) has been trained on the basis of training data (TD),
wherein the training data (TD) for each reference object of a plurality of reference objects comprise as target data: (i) a first reference representation (RR1) of a reference region of the reference object and a second reference representation (RR2) of the reference region of the reference object as input data and (ii) a third reference representation (RR3) of the reference region of the reference object,
wherein the first reference representation (RR1) represents the reference region without contrast agent or after administration of a first amount of a contrast agent,
wherein the second reference representation (RR2) represents the reference region after administration of a second amount of the contrast agent, the second amount being larger than the first amount,
wherein the third reference representation (RR3) represents the reference region after administration of a third amount of the contrast agent, the third amount being different from the second amount,
wherein the trained machine-learning model (M^{T}) comprises two submodels: a first submodel (SM1^{T}) and a second submodel (SM2), wherein the first submodel (SM1^{T}) is configured and has been trained to determine, based on the first reference representation (RR1) and second reference representation (RR2) of an examination region of a reference object, at least one model parameter (MP) for the second submodel (SM2) and wherein the second submodel (SM2) is configured to generate, based on at least one of the first reference representation (RR1) and second reference representation (RR2) and on the at least one model parameter (MP) determined by the first submodel (SM1^{T}), a synthetic third reference representation (RR3*);
provide a first representation (R1), wherein the first representation (R1) represents an examination region of an examination object without contrast agent or after administration of a first amount of a contrast agent;
provide a second representation (R2), wherein the second representation (R2) represents the examination region of the examination object after administration of a second amount of the contrast agent, the second amount being larger than the first amount;
feed the first representation (R1) and the second representation (R2) to the trained machine-learning model (M^{T});
receive from the trained machine-learning model (M^{T}) a synthetic third representation (R3*) of the examination region of the examination object, wherein the synthetic third representation (R3*) represents the reference region after administration of a third amount of the contrast agent, the third amount being different from the second amount; and
output and store the synthetic third representation (R3*) and transmit the synthetic third representation (R3*) to a separate computer system.

18. A contrast agent for use in a radiological examination method, the method comprising:
providing a trained machine-learning model (M^{T});
wherein the trained machine-learning model (M^{T}) has been trained on the basis of training data (TD),
wherein the training data (TD) for each reference object of a plurality of reference objects comprise as target data: (i) a first reference representation (RR1) of a reference region of the reference object and a second reference representation (RR2) of the reference region of the reference object as input data and (ii) a third reference representation (RR3) of the reference region of the reference object,
wherein the first reference representation (RR1) represents the reference region without contrast agent or after administration of a first amount of the contrast agent,
wherein the second reference representation (RR2) represents the reference region after administration of a second amount of the contrast agent, the second amount being larger than the first amount,
wherein the third reference representation (RR3) represents the reference region after administration of a third amount of the contrast agent, the third amount being different from the second amount,
wherein the trained machine-learning model (M^{T}) comprises two submodels: a first submodel (SM1^{T}) and a second submodel (SM2), wherein the first submodel (SM1^{T}) is configured and has been trained to determine, based on the first reference representation (RR1) and second reference representation (RR2) of an examination region of a reference object, at least one model parameter (MP) for the second submodel (SM2) and where the second submodel (SM2) is configured to generate, based on at least one of the first reference representation (RR1) and second reference representation (RR2) and on the at least one model parameter (MP) determined by the first submodel (SM1^{T}), a synthetic third reference representation (RR3*);
providing a first representation (R1), wherein the first representation (R1) represents an examination region of an examination object without contrast agent or after administration of a first amount of the contrast agent;
providing a second representation (R2), wherein the second representation (R2) represents the examination region of the examination object after administration of a second amount of the contrast agent, the second amount being larger than the first amount;
feeding the first representation (R1) and the second representation (R2) to the trained machine-learning model (M^{T});
receiving from the trained machine-learning model (M^{T}) a synthetic third representation (R3*) of the examination region of the examination object, wherein the synthetic third representation (R3*) represents the reference region after administration of a third amount of the contrast agent, the third amount being different from the second amount; and
outputting and storing the synthetic third representation (R3*) and transmitting the synthetic third representation (R3*) to a separate computer system.

19. The contrast agent for use according to claim 18, wherein the radiological examination method is a magnetic resonance imaging examination or a computed tomography examination and wherein the contrast agent comprises:
a Gd³⁺ complex of a compound of formula (I) 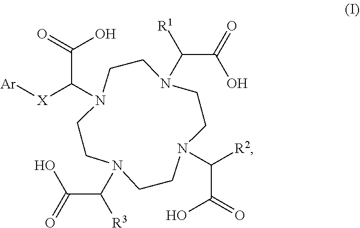
wherein:
Ar is a group selected from: 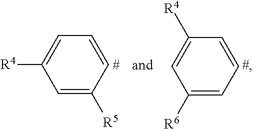
wherein _{#} is a linkage to X;
X is a group selected from:
CH₂, (CH₂)₂, (CH₂)₃, (CH₂)₄ and *—(CH₂)₂O—CH₂—^{#};
wherein * is a linkage to Ar and ^{#} is a linkage to an acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom;
and
R⁶ is a hydrogen atom;
or a stereoisomer, a tautomer, a hydrate, a solvate or a salt thereof, or a mixture thereof, or
a Gd³⁺ complex of a compound of formula (II) 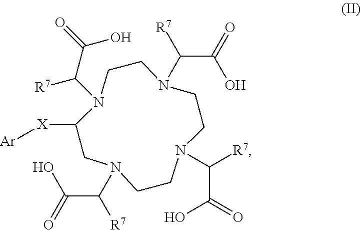
wherein:
Ar is a group selected from: 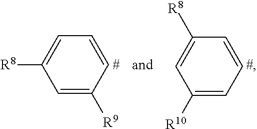
wherein _{#} is a linkage to X;
X is a group selected from CH₂, (CH₂)₂, (CH₂)₃, (CH₂)₄ and *—(CH₂)₂O—CH₂—^{#};
wherein * is a linkage to Ar and ^{#} is a linkage to an acetic acid residue;
R⁷ is a hydrogen atom or a group selected from C₁-C₃ alkyl, —CH₂OH, —(CH₂)₂OH and —CH₂OCH₃;
R⁸ is a group selected from:
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are independently a hydrogen atom;
or a stereoisomer, a tautomer, a hydrate, a solvate or a salt thereof, or a mixture thereof, or the contrast agent comprises one of the following substances:
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(±)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate;
gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium-2,2′,2″-[10-(carboxymethyl)-2-(4-ethoxybenzyl)-1,4,7,10-tetraazacyclododecane-1,4,7-triyl]triacetate.

20. A kit comprising a computer program product according to claim 17 and a contrast agent, wherein the contrast agent comprises:
a Gd³⁺ complex of a compound of formula (I) 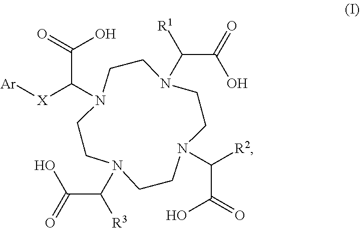
wherein:
Ar is a group selected from: 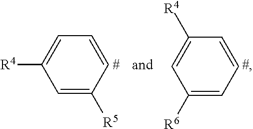
wherein _{#} is a linkage to X;
X is a group selected from:
CH₂, (CH₂)₂, (CH₂)₃, (CH₂)₄ and *—(CH₂)₂O—CH₂—^{#};
wherein * is a linkage to Ar and ^{#} is a linkage to an acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom;
and
R⁶ is a hydrogen atom;
or a stereoisomer, a tautomer, a hydrate, a solvate or a salt thereof, or a mixture thereof, or
a Gd³⁺ complex of a compound of formula (II) 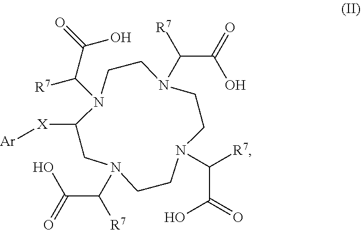
wherein:
Ar is a group selected from: 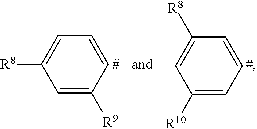
wherein _{#} is a linkage to X;
X is a group selected from CH₂, (CH₂)₂, (CH₂)₃, (CH₂)₄ and *—(CH₂)₂O—CH₂—^{#};
wherein * is a linkage to Ar and ^{#} is a linkage to an acetic acid residue;
R⁷ is a hydrogen atom or a group selected from C₁-C₃ alkyl, —CH₂OH, —(CH₂)₂OH and —CH₂OCH₃;
R⁸ is a group selected from:
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are independently a hydrogen atom;
or a stereoisomer, a tautomer, a hydrate, a solvate or a salt thereof, or a mixture thereof, or the contrast agent comprises one of the following substances:
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(±)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate;
gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium-2,2′,2″-[10-(carboxymethyl)-2-(4-ethoxybenzyl)-1,4,7,10-tetraazacyclododecane-1,4,7-triyl]triacetate.
